(19) United States
(12) Patent Application Publication
(10) Pub. No.: US 2021/0244814 A1
(43) Pub. Date: Aug. 12, 2021

(54) COMBINATION OF METFORMIN AND CYCLOPHOSPHAMIDE AS AN ADJUVANT IN CANCER IMMUNOTHERAPY

(71) Applicant: UNIVERSITÉ CATHOLIQUE DE LOUVAIN, Louvain la Neuve (BE)

(72) Inventors: Benoît VAN DEN EYNDE, Rixensart (BE); Veronica FINISGUERRA, Louvain Brussels (BE)

(73) Assignee: UNIVERSITÉ CATHOLIQUE DE LOUVAIN, Louvain la Neuve (BE)

(21) Appl. No.: 17/049,435

(22) PCT Filed: Apr. 25, 2019
(86) PCT No.: PCT/EP2019/060641

§ 371 Date: Oct. 21, 2020

(30) Foreign Application Priority Data
Apr. 25, 2018 (EP) 18169331.8

(51) Int. Cl. A61K 39/395; A61P 35/00

(52) U.S. Cl. CPC A61K 39/3955; A61K 2039/505; A61P 35/00

(57) ABSTRACT

A combination including metformin and cyclophosphamide for use with an immunotherapy in the treatment of a solid cancer. In particular, the combination including metformin and cyclophosphamide is used as an adjuvant for an immunotherapy. More specifically, a combination including metformin and cyclophosphamide for use with an adoptive cell therapy, with a therapeutic vaccine, with a checkpoint inhibitor therapy or with a T-cell agonist therapy, preferably with an adoptive cell therapy or a checkpoint inhibitor therapy, in the treatment of a solid cancer.

### FIELD OF INVENTION

The present invention relates to the field of cancer therapy, in particular to the field of immunotherapy used for the treatment of cancer. More specifically, the present invention relates to an adjuvant combination comprising metformin and cyclophosphamide for use with an immunotherapy in the treatment of a solid cancer.

### BACKGROUND OF INVENTION

In recent years, immunotherapy has proven to be one of the most promising development in cancer treatment Immunotherapy manipulates a subject immune system with the aim of enhancing the immune response of the subject towards tumor cells, and thus of inducing the specific destruction of the tumor cells.

Currently, immunotherapy in cancer treatment can take many different forms, and includes for example the adoptive transfer of cells, notably of cytotoxic T cells, the administration of checkpoint inhibitors, the administration of T-cell agonists, the administration of monoclonal antibodies or the administration of cytokines (Ribas & Wolchok, 2018, Science 359, 1350-1355; Galluzzi et al., 2014, Oncotarget 5, 12472-12508; Sharma & Allison, 2015, Science 348, 56-61). Immunotherapy in cancer treatment also includes therapeutic vaccines and the use of BCG (Bacillus Calmette-Guérin), the latter being used in the treatment of bladder cancer (Ribas & Wolchok, 2018, Science 359, 1350-1355; Garg et al., 2017, Trends Immunol 38, 577-593; Durgeau et al.,2018, Front Immunol 9, 14).

One of the central premises underlying cancer immunotherapy is the presence of antigens which are selectively or abundantly expressed or mutated in tumor cells, thus enabling the specific recognition and subsequent destruction of the tumor cells (Wirth & Kuhnel, 2017, Front Immunol 8, 1848; Hugo et al., 2016, Cell 165, 35-44). Another of the central premises underlying cancer immunotherapy is the presence of immune cells in the tumors, in particular of lymphocytes (Tumeh et al., 2014, Nature 515, 568-571). Such lymphocytes, commonly referred to as tumor infiltrating lymphocytes (TILs), notably comprise effector TILs which can target and kill the tumor cells through the recognition of the above-mentioned tumor-specific antigens (Durgeau et al.,2018, Front Immunol 9, 14; Tumeh et al., 2014, Nature 515, 568-571).

Yet, depending on the type of cancer and on the individual response, tumors are infiltrated to a varying degree with immune cells, and in particular with lymphocytes. Tumors with a high presence of lymphocytes are commonly referred to as “hot tumors”, while tumors with a low presence of lymphocytes are commonly referred to as “cold tumors” (Sharma & Allison, 2015, Science 348, 56-61).

It is known that increased effector T cell infiltration into tumors, and thus increased T cell response against the tumor cells, is correlated with increased survival for many different types of cancers. Thus, a number of cancer immunotherapies aim at increasing the infiltration and/or the activation of effector T cells within tumors.

One such immunotherapy consists in the transfer, i.e., infusion, of tumor infiltrating T cells to a subject. Such transfer, referred to as an adoptive cell transfer, was first described in 1988 (Rosenberg et al., 1988, N Engl J Med 319, 1676-1680). Later, the introduction of an immunodepleting preparative regimen, to be given to the subject before the adoptive transfer, marked a decisive improvement in efficacy (Rosenberg & Restifo, 2015, Science 348, 62-68). Such regimen indeed allows a clonal repopulation of the subject with tumor-specific T cells. Adoptive cell transfer of tumor infiltrating T cells has been used for example in clinical trials for the treatment of metastatic melanoma, with an objective response observed in approximately 50% of patients (Geukes et al., 2015, Mol Oncol 9, 1918-1935).

Another such immunotherapy consists in the administration of a checkpoint inhibitor. Checkpoint inhibitors block interactions between inhibitory receptors expressed on T cells and their ligands. Checkpoint inhibitors are administered to prevent the inhibition of T cells by factors expressed by tumor cells and thus to enhance the T cell response against said tumor cells (Marin-Acevedo et al., 2018, J Hematol Oncol 11, 39).

However, the overall efficacy of immunotherapy remains limited in the majority of patients (Jenkins et al., 2018, Br J Cancer 118, 9-16; Ladanyi. 2015, Pigment Cell Melanoma Res 28, 490-500). One critical issue is the number of tumor-specific T cells present in the tumor and the exhaustion of said tumor infiltrating T cells, defined as poor effector function, sustained expression of inhibitory receptors and a transcriptional state distinct from that of functional effector or memory T cells (Jochems & Schlom, 2011, Exp Biol Med (Maywood) 236, 567-579).

Tumors represent a hostile microenvironment for T cells. Indeed, the irregular blood flow and endothelial cell (EC) anergy that characterize most solid tumors limit lymphocyte adhesion, extravasation, and infiltration. Moreover, the limited blood supply also results in hypoxia and in the reprograming of energy metabolism within tumor cells, thus transforming the tumor in a harsh environment that limits survival and effector function of T cells (Koppenol et al., 2011, Nat Rev Cancer 11, 325-337; Siska & Rathmell, 2015, Trends Immunol 36, 257-264; Young et al., 2014, Cancer Discov 4, 879-888).

Thus, there is a need for more efficient immunotherapies, characterized by a better infiltration of tumors with tumor-specific T cells and/or by sustained effector function of said T cells within the tumor. In particular, there is still a need for adjuvants to be administered with a cancer immunotherapy, said adjuvants potentiating the immunotherapy notably by improving tumor-specific T cells infiltration in the tumors, survival of the tumor-specific T cells within the tumors and/or effector function of the tumor-specific T cells within the tumors.

The present invention thus relates to a combination comprising metformin and cyclophosphamide for use with an immunotherapy in the treatment of a solid cancer. As illustrated hereinafter, the combination comprising metformin and cyclophosphamide acts as an adjuvant for said immunotherapy and potentiates the effects of said immunotherapy in the treatment of a solid cancer. In particular, the present invention relates to a combination comprising metformin and cyclophosphamide for use with an adoptive cell therapy, with a therapeutic vaccine, with a checkpoint inhibitor therapy or with a T-cell agonist therapy in the treatment of a solid cancer, preferably with an adoptive cell therapy or a checkpoint inhibitor therapy.

### SUMMARY

The present invention relates to a combination comprising metformin and cyclophosphamide for use with an immunotherapy in the treatment of a solid cancer in a subject in need thereof. In one embodiment, the combination for use according to the invention is used as an adjuvant for the immunotherapy.

In one embodiment, the solid cancer to be treated according to the invention is selected from the group comprising melanoma, Merkel cell skin cancer, Hodgkin's Lymphoma, lung cancer, head and neck cancer, bladder cancer, kidney cancer, cervical cancer, pancreatic cancer, prostate cancer, breast cancer, gastric cancer, and glioblastoma. In one embodiment, the solid cancer to be treated according to the invention is selected from the group comprising melanoma, Merkel cell skin cancer, lung cancer, head and neck cancer, bladder cancer, colon cancer, kidney cancer, cervical cancer, pancreatic cancer, prostate cancer, breast cancer, gastric cancer, and glioblastoma.

In one embodiment, metformin and cyclophosphamide are to be administered sequentially.

In one embodiment, metformin is to be administered at a dose ranging from about 0.15 mg per kilo body weight per day (mg/kg/day) to about 150 mg/kg/day, preferably from about 1.5 mg/kg/day to about 100 mg/kg/day, and cyclophosphamide is to be administered at a dose ranging from about 0.15 mg/kg/day to about 150 mg/kg/day, preferably from about 0.7 mg/kg/day to about 100 mg/kg/day.

According to one embodiment, the immunotherapy comprises the adoptive transfer of immune cells or a checkpoint inhibitor.

According to one embodiment, the immunotherapy comprises the adoptive transfer of T cells. In one embodiment, said T cells are CD8⁺ T cells. In one embodiment, said T cells are autologous T cells. In one embodiment, said T cells are CAR T cells.

In one embodiment, metformin and cyclophosphamide are to be administered sequentially, with cyclophosphamide to be administered prior to the adoptive transfer of T cells and metformin to be administered prior to or concomitantly with the adoptive transfer of T cells.

According to one embodiment, the immunotherapy comprises a checkpoint inhibitor. In one embodiment, said checkpoint inhibitor is selected from the group comprising inhibitors of PD-1 such as pembrolizumab, nivolumab, cemiplimab, tislelizumab and spartalizumab, inhibitors of PD-L1 such as avelumab, atezolizumab and durvalumab, inhibitors of CTLA-4 such as ipilimumab and tremelimumab, and any mixtures thereof. In one embodiment, said checkpoint inhibitor is an anti-PD-1 antibody, such as pembrolizumab, nivolumab, cemiplimab, tislelizumab and spartalizumab.

In one embodiment, metformin and cyclophosphamide are to be administered sequentially, with cyclophosphamide to be administered prior to the checkpoint inhibitor and metformin to be administered prior to or concomitantly with the checkpoint inhibitor.

Definitions

In the present invention, the following terms have the following meanings:
“About” preceding a figure encompasses plus or minus 10%, or less, of the value of said figure. It is to be understood that the value to which the term “about” refers is itself also specifically, and preferably, disclosed.
“Adjuvant” in the present invention refers to a compound or combination of compounds that potentiates an immunotherapy used in a solid cancer treatment, and thus potentiates the immune response towards tumors cells. For example, an adjuvant may increase the number of tumor-infiltrated effector lymphocytes, increase the activation of tumor-infiltrated effector lymphocytes, increase the fitness of tumor-infiltrated effector lymphocytes, and/or increase the survival of tumor-infiltrated effector lymphocytes.
“Allogeneic” or “allogenic” refers to any material, in particular any cells, obtained or derived from a different subject of the same species than the subject to whom/which the material is to be introduced. Two or more subjects are said to be allogeneic to one another when the genes at one or more loci are not identical. In some aspects, allogeneic material from subjects of the same species may be sufficiently unlike genetically to interact antigenically.
“Autologous” refers to any material, in particular any cells, obtained or derived from the same subject to whom/which it is later to be re-introduced.
“Cancer immunotherapy” refers to an immunotherapy used for the treatment of a cancer, preferably of a solid cancer, said immunotherapy modulating the immune response of a subject with the aim of enhancing the immune response of the subject towards tumor cells. In one embodiment, “cancer immunotherapy” refers to an immunotherapy used for the treatment of a cancer, preferably of a solid cancer, said immunotherapy stimulating the immune cell response, in particular the T cell response of a subject towards tumor cells. In one embodiment, the cancer immunotherapy comprises or consists of the adoptive transfer of immune cells, preferably T cells, or of the administration of a checkpoint inhibitor. In one embodiment, the cancer immunotherapy comprises or consists of the adoptive transfer of T cells, in particular of effector T cells. In another embodiment, the cancer immunotherapy comprises or consists of the administration of a checkpoint inhibitor, in particular of an inhibitor of PD-1, PD-L1 or CTLA-4, such as an anti-PD-1, anti-PD-L1 or anti-CTLA-4 antibody.
“Combination” in the present invention refers to the association of two compounds, e.g., metformin and cyclophosphamide, to be administered to the same subject. According to the present invention, said two compounds can be administered concomitantly or sequentially. Thus, according to the present invention, the administration of the first compound does not necessarily overlap with the administration of the second compound.
“MDSCs” refers to myeloid-derived suppressor cells, which represent a heterogeneous population of immature myeloid cells with suppressive activity, comprising precursors of granulocytes, macrophages, and dendritic cells. MDSCs are elevated in virtually all patients and experimental mice with malignancies.
“Pharmaceutically acceptable excipient” or “pharmaceutically acceptable carrier” refers to an excipient or carrier that does not produce an adverse, allergic or other untoward reaction when administered to a mammal, preferably a human. It includes any and all solvents, dispersion media, coatings, antibacterial and antifungal agents, isotonic and absorption delaying agents and the like. A pharmaceutically acceptable carrier or excipient refers to a non-toxic solid, semi-solid or liquid filler, diluent, encapsulating material or formulation auxiliary of any type. For human administration, preparations should meet sterility, pyrogenicity, general safety and purity standards as required by the regulatory offices such as the FDA or EMA.
“Pharmaceutically acceptable salt” refers to salts of a free acid or a free base which are not biologically undesirable and are generally prepared by reacting the free base with a suitable organic or inorganic acid or by reacting the free acid with a suitable organic or inorganic base.
“Subject” refers to a mammal, preferably a human. In one embodiment, the subject is diagnosed with a solid cancer. In one embodiment, the subject is a patient, preferably a human patient, who/which is awaiting the receipt of, or is receiving, medical care or was/is/will be the subject of a medical procedure or is monitored for the development or progression of a disease. In one embodiment, the subject is a human patient who is treated and/or monitored for the development or progression of a solid cancer. In one embodiment, the subject is a male. In another embodiment, the subject is a female. In one embodiment, the subject is an adult. In another embodiment, the subject is a child.
“T cell fitness” as used herein is assessed through the TCR-triggered signaling, proliferation, cytokine production, and/or survival of T cells. Thus, in one embodiment, a poor T cell fitness refers to a decrease capacity of T cells for TCR-triggered signaling, proliferation and/or cytokine production, and/or to a decreased survival of T cells. In another embodiment, an increased T cell fitness refers to an increased capacity of T cells for TCR-triggered signaling, proliferation and/or cytokine production, and/or to an increased survival of T cells.
“T cell response” as used herein is defined as a T cell mediated immune response of a subject, in particular a T cell mediated immune response towards the tumor cells of a subject suffering from a solid cancer.
“Tumor infiltrating T cell response” as used herein is defined as an immune response towards tumor cells of a subject suffering from a solid cancer, the immune response being mediated by tumor infiltrating T cells, i.e., T cells present in the tumor(s).
“Therapeutically effective amount” or “therapeutically effective dose” refer to the amount or dose of metformin, to the amount or dose of cyclophosphamide, or to the amount or dose of both, in a combination according to the invention, that is aimed at, without causing significant negative or adverse side effects to the subject, (1) delaying or preventing the onset of a solid cancer in the subject; (2) reducing the severity or incidence of a solid cancer; (3) slowing down or stopping the progression, aggravation, or deterioration of one or more symptoms of a solid cancer affecting the subject; (4) bringing about ameliorations of the symptoms of a solid cancer affecting the subject; or (5) curing a solid cancer affecting the subject. A therapeutically effective amount may be administered prior to the onset of a solid cancer for a prophylactic or preventive action. Alternatively, or additionally, a therapeutically effective amount may be administered after initiation of a solid cancer, for a therapeutic action.
“Treating” or “treatment” refers to therapeutic treatment, to prophylactic or preventative measures, or to both, wherein the object is to prevent or slow down (lessen) the targeted pathologic condition or disorder, i.e., a solid cancer. Those in need of treatment include those already suffering from a solid cancer as well as those prone to develop a solid cancer or those in whom solid cancer is to be prevented. A subject is successfully “treated” if, after receiving a therapeutic amount of metformin and of cyclophosphamide in combination according to the present invention, the subject shows observable and/or measurable reduction in the number of cancer cells (also referred to as tumor cells); reduction in the percent of total cells that are cancerous (i.e., reduction of the tumor(s)) ; relief to some extent of one or more of the symptoms associated with the solid cancer; reduced morbidity and mortality, and/or improvement in quality of life issues. The above parameters for assessing successful treatment and improvement in the disease are readily measurable by routine procedures familiar to a physician.
“Tumor infiltrating lymphocytes” or “TILs” refer to the lymphocytes, in particular the T cells, that are present in a tumor, either before an immunotherapy or after an immunotherapy, such as for example adoptive cell transfer or therapeutic vaccines. As used herein, lymphocytes encompass natural killer (NK) cells and T cells. As used herein, T cells encompass CD4⁺ T cells and CD8⁺ T cells. As used herein, T cells also encompass T regulatory (Treg) cells, such as CD4⁺ Treg cells or CD8⁺T reg cells, and T effector cells, such as CD4⁺ effector T cells and CD8⁺ effector T cells. In particular, CD8⁺ effector T cells include cytotoxic CD8⁺ T cells. In one embodiment, effector tumor infiltrating lymphocytes, or effector TILs, are the CD4⁺ or CD8⁺ effector T cells present in a tumor, either before an immunotherapy or after an immunotherapy, such as for example adoptive cell transfer or therapeutic vaccines. In one embodiment, regulatory tumor infiltrating lymphocytes, or regulatory TILs, are the CD4⁺ or CD8⁺ Treg cells present in a tumor, either before an immunotherapy or after an immunotherapy, such as for example adoptive cell transfer or therapeutic vaccines.
“Tumor-specific antigen” or “tumor-associated antigen” refers to an antigen specifically and/or abundantly expressed by tumor cells also referred to as cancer cells.

### DETAILED DESCRIPTION

The present invention thus relates to a combination comprising metformin and cyclophosphamide for use with an immunotherapy in the treatment of a solid cancer in a subject in need thereof. The present invention also relates to a combination consisting of metformin and cyclophosphamide for use with an immunotherapy in the treatment of a solid cancer in a subject in need thereof.

In particular, the present invention relates to a combination comprising or consisting of metformin and cyclophosphamide for use with an immunotherapy in the treatment of a solid cancer in a subject in need thereof, said combination being used as an adjuvant for the immunotherapy. In one embodiment, the present invention relates to a combination comprising or consisting of metformin and cyclophosphamide for use in the treatment of a solid cancer in a subject in need thereof, said combination being used as an adjuvant for an immunotherapy.

The present invention also relates to an adjuvant for an immunotherapy for the treatment of a solid cancer comprising or consisting of metformin and cyclophosphamide. In other words, the present invention also relates to an adjuvant for a solid cancer immunotherapy comprising or consisting of metformin and cyclophosphamide.

The Applicant surprisingly showed that the combined administration of metformin and cyclophosphamide significantly potentiates an immunotherapy used in the treatment of a solid cancer. As illustrated in the Examples hereinafter, the combined administration of metformin and cyclophosphamide increased the infiltration and survival of cytotoxic T cells in tumors upon adoptive cell therapy, and thus resulted in an inhibition of tumor growth and an increase of mice survival. As illustrated in the Examples hereinafter, the combined administration of metformin and cyclophosphamide also increased tumor response to a PD-1 inhibitor. The Applicant thus showed that metformin and cyclophosphamide act in synergy as an adjuvant for an immunotherapy, for example an adoptive cell therapy or a checkpoint inhibitor therapy, used in the treatment of a solid cancer. Indeed, the effects of a combination of metformin and cyclophosphamide were higher than the individual effects of metformin, than the individual effects of cyclophosphamide and higher than the sum of the two individual effects.

Metformin (CAS number 1115-70-4) is also known as 3-(diaminomethylidene)-1,1-dimethylguanidine. Other names used to refer to metformin include 1,1-dimethylbiguanide and dimethylguanylguanidine. Trade names of metformin include, without being limited to, Glucophage®, Glumetza® and Fortamet®. Metformin is also commonly referred to as MTF.

Metformin has the following formula:

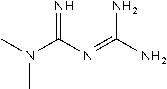

As used herein, the term “metformin” encompasses any prodrugs, pharmaceutically acceptable salts, hydrates and solvates thereof. In particular, the term “metformin” encompasses the hydrochloride salts and mono-hydrochloride salt thereof, e.g., metformin hydrochloride and metformin mono-hydrochloride. The term “metformin” also encompasses the crystalline forms of said compound.

More generally, metformin belongs to the biguanides, which also include, without being limited to, phenformin and buformin Biguanide compound are mainly known as antihyperglycemic agents. Some biguanides such as proguanil are also used as antimalarial agents.

In one embodiment, metformin as described hereinabove is replaced by another biguanide compound. In one embodiment, metformin is replaced by phenformin or by buformin. Thus, in one embodiment, the present invention also relates to a combination comprising or consisting of cyclophosphamide and one of metformin, phenformin, or buformin for use with an immunotherapy in the treatment of a solid cancer in a subject in need thereof.

Metformin is widely used as an oral anti-hyperglycemic agent in the treatment of type 2 diabetes. Metformin induces a suppression of hepatic gluco-neogenesis and an improved insulin sensitivity. Said effects are believed to be exerted through an indirect activation of AMPK following the inhibition of the complex I of the mitochondrial respiratory chain. Metformin inhibits ATP synthesis and, thus, increases intracellular levels of ADP and AMP which leads to the activation of AMPK via LKB1 activation.

Cyclophosphamide (CAS number 6055-19-2) is also known as 2-[bis(2-chloroethyl)amino]-1,3,2λ⁵-oxazaphosphinan-2-one. Other names used to refer to cyclophosphamide include 2-[Bis(2-chloroethylamino)]-tetrahydro-2H-1,3,2-oxazaphosphorine-2-oxide and Bis(2-chloroethyl)phosphoramide cyclic propanolamide ester. Trade names of cyclophosphamide include, without being limited to, Cytoxan®, Neosar®, and Procytox®. Cyclophosphamide is also commonly referred to as CTX or CPA.

Cyclophosphamide has the following formula:

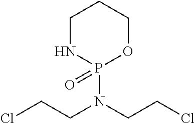 [SMILES: O=P1(N(CCCl)CCCl)NCCCO1]

As used herein, the term “cyclophosphamide” encompasses any prodrugs, pharmaceutically acceptable salts, hydrates and solvates thereof. In particular, the term “cyclophosphamide” encompasses monohydrate forms thereof, i.e., cyclophosphamide monohydrate. The term “cyclophosphamide” also encompasses the crystalline forms of said compound.

As used herein, the term “cyclophosphamide” encompasses any stereoisomer thereof, in particular (R)-cyclophosphamide and (S)-cyclophosphamide.

Cyclophosphamide is a synthetic compound chemically related to the nitrogen mustards. More specifically, cyclophosphamide is a prodrug of a nitrogen mustard alkylating agent, in which the reactivity of the bis(2-chloroethyl)amino group is attenuated. Cyclophosphamide requires metabolization by specific liver CYP enzymes to become therapeutically active. Upon oxidation in vivo the six-membered ring opens, enhancing the reactivity of the nitrogen mustard, which then acts to cross-link DNA, thus leading to the inhibition of DNA replication and apoptosis.

More generally, cyclophosphamide belongs to the oxazaphosphorines, which also include, without being limited to, ifosfamide and trofosfamide (also referred to as trophosphamide). As mentioned hereinabove, oxazaphosphorines are alkylating prodrugs which require metabolic activation by the liver.

In one embodiment, cyclophosphamide as described hereinabove is replaced by another oxazaphosphorine. In one embodiment, cyclophosphamide is replaced by ifosfamide or by trofosfamide. Thus, in one embodiment, the present invention also relates to a combination comprising or consisting of metformin and one of cyclophosphamide, ifosfamide, or trofosfamide for use with an immunotherapy in the treatment of a solid cancer in a subject in need thereof.

The present invention thus relates to a combination comprising metformin as described hereinabove and cyclophosphamide as described hereinabove for use with an immunotherapy in the treatment of a solid cancer in a subject in need thereof.

In one embodiment, the present invention relates to a combination consisting of metformin as described hereinabove and cyclophosphamide as described hereinabove for use with an immunotherapy in the treatment of a solid cancer in a subject in need thereof.

In another embodiment, the present invention relates to a combination of metformin as described hereinabove and cyclophosphamide as described hereinabove for use with an immunotherapy in the treatment of a solid cancer in a subject in need thereof.

According to the present invention, metformin and cyclophosphamide in a combination of the invention are to be administered either simultaneously, separately or sequentially with respect to each other.

Another object of the present invention is a kit-of-parts comprising a first part comprising metformin and a second part comprising cyclophosphamide for use in the treatment of a solid cancer in a subject in need thereof, wherein metformin and cyclophosphamide are used as an adjuvant for an immunotherapy.

In one embodiment, the kit-of parts for use in the treatment of a solid cancer in a subject in need thereof according to the invention comprises a first part comprising metformin, a second part comprising cyclophosphamide, and a third part comprising an immunotherapy, such as, for example, a checkpoint inhibitor.

According to the present invention, an immunotherapy is defined as a therapy modulating the immune response of a subject. In a particular embodiment, an immunotherapy as a cancer treatment, i.e., a cancer immunotherapy, is defined as a therapy modulating the immune response of a subject with the aim of enhancing the immune response of the subject towards tumor cells.

According to one embodiment, an immunotherapy as a cancer treatment, i.e., a cancer immunotherapy, is defined as a therapy stimulating the immune response of a subject, in particular the immune response towards tumor cells.

In one embodiment, an immunotherapy as a cancer treatment, i.e., a cancer immunotherapy, is defined as a therapy stimulating the lymphocyte-mediated immune response of a subject, in particular the lymphocyte-mediated immune response towards tumor cells. In one embodiment, an immunotherapy as a cancer treatment, i.e., a cancer immunotherapy, is defined as a therapy stimulating the tumor infiltrating lymphocyte (TIL) response of a subject.

In one embodiment, an immunotherapy as a cancer treatment, i.e., a cancer immunotherapy, is defined as a therapy stimulating the T cell response of a subject, in particular the T cell response towards tumor cells. In one embodiment, an immunotherapy as a cancer treatment, i.e., a cancer immunotherapy, is defined as a therapy stimulating the tumor infiltrating T cell response of a subject.

According to one embodiment, an immunotherapy as a cancer treatment, i.e., a cancer immunotherapy, is defined as a therapy stimulating the immune response of a subject towards tumor cells. Thus, in one embodiment, the immunotherapy of the present invention targets tumor cells. In other words, in one embodiment, the immunotherapy of the present invention aims to/is for killing tumor cells. According to one embodiment, tumor cells are not lymphocytes. Thus, in one embodiment, the immunotherapy of the present invention does not aim to/is not for killing lymphocytes.

One of the central premises underlying cancer immunotherapy is the presence of antigens which are selectively or abundantly expressed or mutated in tumor cells, thus enabling the specific recognition and subsequent destruction of the tumor cells. Another of the central premises underlying cancer immunotherapy is the presence of lymphocytes in the tumors, i.e., tumor infiltrating lymphocytes (TILs), and notably of effector TILs which can target and kill the tumor cells through the recognition of the above-mentioned tumor-specific antigens.

Examples of cancer immunotherapy include, without being limited to, adoptive transfer of cells, checkpoint inhibitors, T-cell agonists, monoclonal antibodies, cytokines, therapeutic vaccines, BCG (Bacillus Calmette-Guérin).

According to one embodiment, the immunotherapy used for the treatment of a solid cancer with a combination comprising or consisting of metformin and cyclophosphamide as described hereinabove comprises or consists of an adoptive transfer of cells also referred to as adoptive cell therapy (both also referred to as ACT), particularly of the adoptive transfer of T cells or NK cells also referred to as adoptive T cell therapy or adoptive NK cell therapy, respectively.

As used herein, an adoptive transfer of cells or adoptive cell therapy is defined as the transfer, for example as an infusion, of immune cells to a subject. As a cancer treatment, the adoptive transfer of immune cells to a subject aims at enhancing the subject immune response towards the tumor cells.

In one embodiment, the transferred immune cells are T cells or natural killer (NK) cells.

In one embodiment, the transferred immune cells are cytotoxic cells. Examples of cytotoxic cells include natural killer (NK) cells, CD8⁺ T cells, and natural killer (NK) T cells.

In one embodiment, the transferred immune cells are natural killer (NK) cells.

In one embodiment, the transferred T cells are T cells, in particular effector T cells. Examples of effector T cells include CD4⁺ T cells and CD8⁺ T cells.

In one embodiment, the transferred immune cells are alpha beta (αβ) T cells. In another embodiment, the transferred immune cells are gamma delta (γδ) T cells.

In one embodiment, the transferred T cells are CD4⁺ T cells, CD8⁺ T cells, or natural killer T cells, preferably the transferred T cells are CD8⁺ T cells.

In one embodiment, the transferred immune cells, in particular T cells, are antigen-specific immune cells, in particular antigen-specific T cells. In one embodiment, said antigen is specifically and/or abundantly expressed by tumor cells. Thus, in one embodiment, the transferred immune cells, in particular T cells, are tumor-specific immune cells, in particular tumor-specific T cells, in other words the transferred immune cells, in particular T cells, specifically recognize tumor cells through an antigen specifically and/or abundantly expressed by said tumor cells. In one embodiment, the transferred immune cells are tumor-specific NK cells. In one embodiment, the transferred immune cells are tumor-specific T cells. In one embodiment, the transferred T cells are tumor-specific effector T cells. In one embodiment, the transferred T cells are tumor-specific CD8⁺ effector T cells, in particular tumor-specific cytotoxic CD8⁺ T cells.

Examples of tumor-specific antigens, i.e., antigens that are specifically and/or abundantly expressed by tumor cells include, without being limited to, neoantigens (also referred to as new antigens or mutated antigens), 9D7, ART4, β-catenin, BING-4, Bcr-abl, BRCA1/2, calcium-activated chloride channel 2, CDK4, CEA (carcinoembryonic antigen), CML66, Cyclin B1, CypB, EBV (Epstein-Barr virus) associated antigens such as LMP-1, LMP-2, EBNA1 and BARF1, Ep-CAM, EphA3, fibronectin, Gp100/pme117, Her2/neu, HPV (human papillomavirus) E6, HPV E7, hTERT, IDH1, IDH2, immature laminin receptor, MC1R, Melan-A/MART-1, MART-2, mesothelin, MUC1, MUC2, MUM-1, MUM-2, MUM-3, NY-ESO-1/LAGE-1, p53, PRAME, prostate-specific antigen (PSA), Ras, SAP-1, SART-I, SART-2, SART-3, SSX-2, survivin, TAG-72, telomerase, TGF-βRII, TRP-1/-2, tyrosinase, WT1, antigens of the BAGE family, antigens of the CAGE family, antigens of the GAGE family, antigens of the MAGE family, antigens of the SAGE family, and antigens of the XAGE family

As used herein, neoantigens (also referred to as new antigens or mutated antigens) correspond to antigens derived from proteins that are the result of somatic mutations or gene rearrangements acquired by the tumors. Neoantigens may be specific to each individual subject and thus provide targets for developing personalized immunotherapies. Examples of neoantigens include for example, without being limited to, the R24C mutant of CDK4, the R24L mutant of CDK4, KRAS mutated at codon 12, mutated p53, the V599E mutant of BRAF and the R132H mutant of IDH1.

In one embodiment, the transferred immune cells, in particular T cells, as described hereinabove are specific for a tumor antigen selected from the group comprising or consisting of the class of CTAs (cancer/testis antigens, also known as MAGE-type antigens), the class of neoantigens and the class of viral antigens.

As used herein, the class of CTAs correspond to antigens encoded by genes that are expressed in tumor cells but not in normal tissues except in male germline cells. Examples of CTAs include, without being limited to, MAGE-A1, MAGE-A3, MAGE-A4, MAGE-C2, NY-ESO-1, PRAME and SSX-2.

As used herein, the class of viral antigens correspond to antigens derived from viral oncogenic proteins. Examples of viral antigens include, without being limited to, HPV (human papillomavirus) associated antigens such as E6 and E7, and EBV (Epstein-Barr virus) associated antigens such as LMP-1, LMP-2, EBNA1 and BARF1.

In one embodiment, the transferred immune cells, in particular T cells, are autologous cells. In another embodiment, the transferred immune cells, in particular T cells, are allogeneic cells.

For example, autologous T cells can be generated ex vivo either by expansion of antigen-specific T cells isolated from the subject or by redirection of T cells of the subject through genetic engineering.

Methods to isolate T cells from a subject in particular antigen-specific T cells, e.g., tumor-specific T cells, are well-known in the art (see for example Rosenberg & Restifo, 2015, Science 348, 62-68; Prickett et al., 2016, Cancer Immunol Res 4, 669-678; or Hinrichs & Rosenberg, 2014, Immunol Rev 257, 56-71). Methods to expand T cells ex vivo are well-known in the art (see for example Rosenberg & Restifo, 2015, Science 348, 62-68; Prickett et al., 2016, Cancer Immunol Res 4, 669-678; or Hinrichs & Rosenberg, 2014, Immunol Rev 257, 56-71). Protocols for infusion of T cells in a subject, including pre-infusion conditioning regimens, are well-known in the art (see for example Rosenberg & Restifo, 2015, Science 348, 62-68; Prickett et al., 2016, Cancer Immunol Res 4, 669-678; or Hinrichs & Rosenberg, 2014, Immunol Rev 257, 56-71).

In one embodiment, the immunotherapy used for the treatment of a solid cancer with a combination comprising or consisting of metformin and cyclophosphamide as described hereinabove comprises or consists of a CAR immune cell therapy, in particular a CAR T cell therapy (Androulla & Lefkothea, 2018, Curr Pharm Biotechnol).

As used herein, CAR immune cell therapy is an adoptive cell therapy wherein the transferred cells are immune cells as described hereinabove, such as NK cells or T cells, genetically engineered to express a chimeric antigen receptor (CAR). As used herein, CAR T cell therapy is an adoptive cell therapy wherein the transferred cells are T cells as described hereinabove genetically engineered to express a chimeric antigen receptor (CAR). As a cancer treatment, the adoptive transfer of CAR immune cells, in particular T cells, to a subject aims at enhancing the subject immune response towards the tumor cells.

CARs are synthetic receptors consisting of a targeting moiety that is associated with one or more signaling domains in a single fusion molecule or in several molecules. In general, the binding moiety of a CAR consists of an antigen-binding domain of a single-chain antibody (scFv), comprising the light and variable fragments of a monoclonal antibody joined by a flexible linker. Binding moieties based on receptor or ligand domains have also been used successfully. The signaling domains for first generation CARs are usually derived from the cytoplasmic region of the CD3zeta or the Fc receptor gamma chains. First generation CARs have been shown to successfully redirect T cell cytotoxicity, however, they failed to provide prolonged expansion and anti-tumor activity in vivo. Thus, signaling domains from co-stimulatory molecules including CD28, OX-40 (CD134), and 4-1BB (CD137) have been added alone (second generation) or in combination (third generation) to enhance survival and increase proliferation of CAR modified T cells.

Thus, in one embodiment, the transferred T cells as described hereinabove are CAR T cells. The expression of a CAR allows the T cells to be redirected against a selected antigen, such as an antigen expressed at the surface of tumor cells. In one embodiment, the transferred CAR T cells recognize a tumor-specific antigen.

Examples of tumor-specific antigens are mentioned hereinabove.

In one embodiment, the transferred CAR T cells recognize a tumor-specific antigen selected from the group comprising or consisting of EGFR and in particular EGFRvIII, mesothelin, PSMA, PSA, CD47, CD70, CD133, CD171, CEA, FAP, GD2, HER2, IL-13Rα, αvβ6 integrin, ROR1, MUC1, GPC3, and EphA2.

According to one embodiment, the immunotherapy used for the treatment of a solid cancer with a combination comprising or consisting of metformin and cyclophosphamide as described hereinabove comprises or consists of at least one checkpoint inhibitors. Thus, in one embodiment, the immunotherapy used for the treatment of a solid cancer with a combination comprising or consisting of metformin and cyclophosphamide as described hereinabove is a checkpoint inhibitor therapy.

As used herein, a checkpoint inhibitor therapy is defined as the administration of at least one checkpoint inhibitor to the subject.

Checkpoint inhibitors (CPI, that may also be referred to as immune checkpoint inhibitors or ICI) block the interactions between inhibitory receptors expressed on T cells and their ligands. As a cancer treatment, checkpoint inhibitor therapy aims at preventing the activation of inhibitory receptors expressed on T cells by ligands expressed by the tumor cells. Checkpoint inhibitor therapy thus aims at preventing the inhibition of T cells present in the tumor, i.e., tumor infiltrating T cells, and thus at enhancing the subject immune response towards the tumor cells.

According to the present invention, a checkpoint inhibitor therapy is defined as a therapy preventing the inhibition of T cells, in particular tumor infiltrating T cells, and maintaining or enhancing the activation of T cells, in particular tumor infiltrating T cells. Thus, according to the present invention, a checkpoint inhibitor therapy is a therapy stimulating the T cell response of a subject, in particular the tumor infiltrating T cell response of a subject.

Examples of checkpoint inhibitors include, without being limited to, inhibitors of the cell surface receptor PD-1 (programmed cell death protein 1), also known as CD279 (cluster differentiation 279); inhibitors of the ligand PD-L1 (programmed death-ligand 1), also known as CD274 (cluster of differentiation 274) or B7-H1 (B7 homolog 1); inhibitors of the cell surface receptor CTLA4 or CTLA-4 (cytotoxic T-lymphocyte-associated protein 4), also known as CD152 (cluster of differentiation 152); inhibitors of LAG-3 (lymphocyte-activation gene 3), also known as CD223 (cluster differentiation 223); inhibitors of TIM-3 (T-cell immunoglobulin and mucin-domain containing-3), also known as HAVCR2 (hepatitis A virus cellular receptor 2) or CD366 (cluster differentiation 366); inhibitors of TIGIT (T cell immunoreceptor with Ig and ITIM domains), also known as VSIG9 (V-Set And Immunoglobulin Domain-Containing Protein 9) or VSTM3 (V-Set And Transmembrane Domain-Containing Protein 3); inhibitors of BTLA (B and T lymphocyte attenuator), also known as CD272 (cluster differentiation 272); inhibitors of CEACAM-1 (carcinoembryonic antigen-related cell adhesion molecule 1) also known as CD66a (cluster differentiation 66a).

In one embodiment, the at least one checkpoint inhibitor is selected from the group comprising or consisting of inhibitors or PD-1, inhibitors of PD-L1, inhibitors of CTLA-4 and any mixtures thereof. Thus, in one embodiment, the checkpoint inhibitor therapy is a PD-1, PD-L1, and/or CTLA-4 inhibitor therapy. In one embodiment, the at least one checkpoint inhibitor is selected from the group comprising or consisting of anti-PD-1 antibodies, anti-PD-L1 antibodies, anti-CTLA-4 antibodies and any mixtures thereof.

In one embodiment, the at least one checkpoint inhibitor is selected from the group comprising or consisting of pembrolizumab, nivolumab, cemiplimab, tislelizumab, spartalizumab, BI 754091, MAG012, TSR-042, AGEN2034, avelumab, atezolizumab, durvalumab, LY3300054, ipilimumab, tremelimumab, and any mixtures thereof.

In one embodiment, the at least one checkpoint inhibitor is selected from the group comprising or consisting of pembrolizumab, nivolumab, cemiplimab, tislelizumab, spartalizumab, avelumab, atezolizumab, durvalumab, ipilimumab, tremelimumab, and any mixtures thereof.

In one embodiment, the at least one checkpoint inhibitor is an inhibitor of PD-1, also referred to as an anti-PD-1. Thus, in one embodiment, the checkpoint inhibitor therapy is a PD-1 inhibitor therapy.

Inhibitors of PD-1 may include antibodies targeting PD-1, in particular monoclonal antibodies, and non-antibody inhibitors such as small molecule inhibitors.

Examples of inhibitors of PD-1 include, without being limited to, pembrolizumab, nivolumab, cemiplimab, tislelizumab, spartalizumab, BI 754091, MAG012, TSR-042, AGEN2034.

Pembrolizumab is also known as MK-3475, MK03475, lambrolizumab, or SCH-900475. The trade name of pembrolizumab is Keytruda®.

Nivolumab is also known as ONO-4538, BMS-936558, MDX1106, or GTPL7335. The trade name of nivolumab is Opdivo®.

Cemiplimab is also known as REGN2810 or REGN-2810.

Tislelizumab is also known as BGB-A317.

Spartalizumab is also known as PDR001 or PDR-001.

In one embodiment, the at least one checkpoint inhibitor is an anti-PD-1 antibody.

In one embodiment, the at least one checkpoint inhibitor is selected from the group comprising or consisting of pembrolizumab, nivolumab, cemiplimab, tislelizumab, spartalizumab, BI 754091, MAG012, TSR-042, AGEN2034, and any mixtures thereof.

In one embodiment, the at least one checkpoint inhibitor is selected from the group comprising or consisting of pembrolizumab, nivolumab, cemiplimab, tislelizumab, spartalizumab, and any mixtures thereof.

In one embodiment, the at least one checkpoint inhibitor is an inhibitor of PD-L1, also referred to as an anti-PD-L1. Thus, in one embodiment, the checkpoint inhibitor therapy is a PD-L1 inhibitor therapy.

Inhibitors of PD-L1 may include antibodies targeting PD-L1, in particular monoclonal antibodies, and non-antibody inhibitors such as small molecule inhibitors.

Examples of inhibitors of PD-L1 include, without being limited to, avelumab, atezolizumab, durvalumab and LY3300054.

Avelumab is also known as MSB0010718C, MSB-0010718C, MSB0010682, or MSB-0010682. The trade name of avelumab is Bavencio®.

Atezolizumab is also known as MPDL3280A (clone YW243.55.S70), MPDL-3280A, RG-7446 or RG7446. The trade name of atezolizumab is Tecentriq®.

Durvalumab is also known as MEDI4736 or MEDI-4736. The trade name of durvalumab is Imfinzi®.

In one embodiment, the at least one checkpoint inhibitor is an anti-PD-L1 antibody.

In one embodiment, the at least one checkpoint inhibitor is selected from the group comprising or consisting of avelumab, atezolizumab, durvalumab, LY3300054, and any mixtures thereof.

In one embodiment, the at least one checkpoint inhibitor is selected from the group comprising or consisting of avelumab, atezolizumab, durvalumab, and any mixtures thereof.

In one embodiment, the at least one checkpoint inhibitor is an inhibitor of CTLA-4, also referred to as an anti-CTLA-4. Thus, in one embodiment, the checkpoint inhibitor therapy is a CTLA-4 inhibitor therapy.

Inhibitors of CTLA-4 may include antibodies targeting CTLA-4, in particular monoclonal antibodies, and non-antibody inhibitors such as small molecule inhibitors.

Examples of inhibitors of CTLA-4 include, without being limited to, ipilimumab and tremelimumab.

Ipilimumab is also known as BMS-734016, MDX-010, or MDX-101. The trade name of ipilimumab is Yervoy®.

Tremelimumab is also known as ticilimumab, CP-675, or CP-675,206.

In one embodiment, the at least one checkpoint inhibitor is an anti-CTLA-4 antibody.

In one embodiment, the at least one checkpoint inhibitor is selected from the group comprising or consisting of ipilimumab, tremelimumab, and any mixtures thereof.

According to one embodiment, the immunotherapy used for the treatment of a solid cancer with a combination comprising or consisting of metformin and cyclophosphamide as described hereinabove comprises or consists of at least one T-cell agonist (sometimes also referred to as checkpoint agonist). Thus, in one embodiment, the immunotherapy used for the treatment of a solid cancer with a combination comprising or consisting of metformin and cyclophosphamide as described hereinabove is a T-cell agonist therapy.

As used herein, a T-cell agonist therapy is defined as the administration of at least one T-cell agonist to the subject.

T-cell agonists act by activating stimulatory receptors expressed on immune cells, such as T cells. As used herein, the term “stimulatory receptors” refer to receptors that induce a stimulatory signal upon activation, and thus lead to an enhancement of the immune response. As a cancer treatment, T-cell agonist therapy aims at activating stimulatory receptors expressed on immune cells present in the tumor. In particular, T-cell agonist therapy aims at enhancing the activation of T cells present in the tumor, i.e., tumor infiltrating T cells, and thus at enhancing the subject immune response towards the tumor cells.

According to one embodiment, a T-cell agonist therapy is thus defined as a therapy stimulating the T cell response of a subject, in particular the tumor infiltrating T cell response of a subject.

Examples of T-cell agonists include, without being limited to, agonists of CD137 (cluster differentiation 137) also known as 4-1BB or TNFRS9 (tumor necrosis factor receptor superfamily, member 9); agonists of OX40 receptor also known as CD134 (cluster differentiation 134) or TNFRSF4 (tumor necrosis factor receptor superfamily, member 4).

In one embodiment, the at least one T-cell agonist is selected from the group comprising or consisting of agonists of CD137, agonists of OX40 and any mixtures thereof.

Examples of agonists of CD137 include, without being limited, utomilumab and urelumab.

According to one embodiment, the immunotherapy used for the treatment of a solid cancer with a combination comprising or consisting of metformin and cyclophosphamide as described hereinabove comprises or consists of a therapeutic vaccine (sometimes also referred to as a treatment vaccine).

As used herein, a therapeutic vaccine is defined as the administration of at least one tumor-specific antigen (e.g., synthetic long peptides or SLP), or of the nucleic acid encoding said tumor-specific antigen; the administration of recombinant viral vectors selectively entering and/or replicating in tumor cells; the administration of tumor cells; and/or the administration of immune cells (e.g., dendritic cells) engineered to present tumor-specific antigens and trigger an immune response against these antigens.

As a cancer treatment, therapeutic vaccines aim at enhancing the subject immune response towards the tumor cells.

Examples of therapeutic vaccines aiming at enhancing the subject immune response towards the tumor cells include, without being limited to, viral-vector based therapeutic vaccines such as adenoviruses (e.g., oncolytic adenoviruses), vaccinia viruses (e.g., modified vaccinia Ankara (MVA)), alpha viruses (e.g., Semliki Forrest Virus (SFV)), measles virus, Herpes simplex virus (HSV), and coxsackievirus; synthetic long peptide (SLP) vaccines; and dendritic cell vaccines.

According to one embodiment, the immunotherapy used for the treatment of a solid cancer with a combination comprising or consisting of metformin and cyclophosphamide as described hereinabove comprises or consists of an antibody therapy.

As used herein, an antibody therapy is defined as the administration of at least one tumor-specific antibody, in particular of a tumor-specific monoclonal antibody, to the subject.

Examples of tumor-specific antibodies, include, without being limited to, antibodies targeting a checkpoint receptor or ligand as described hereinabove, antibodies targeting a cell surface markers of tumor cells, and antibodies targeting proteins involved in the growth or spreading of tumor cells.

As a cancer treatment, antibody therapy aims at enhancing the subject immune response towards the tumor cells, notably by targeting tumor cells for destruction, by stimulating the activation of T cells present in the tumor or by preventing the inhibition of T cells present in the tumor, or at inhibiting the growth or spreading of tumor cells.

According to one embodiment, the antibody therapy of the present invention enhances the subject immune response towards the tumor cells by stimulating the T cell response of a subject, in particular the tumor infiltrating T cell response of a subject.

Examples of antibodies, in particular monoclonal antibodies, preventing the inhibition of T cells present in the tumor include, without being limited to, anti-PD-1 antibodies (such as pembrolizumab, nivolumab, cemiplimab, tislelizumab, and spartalizumab), anti-PD-L1 antibodies (such as avelumab, atezolizumab, and durvalumab) and anti-CTLA-4 antibodies (such as ipilimumab and tremelimumab) as described hereinabove.

Examples of antibodies, in particular monoclonal antibodies, stimulating the activation of T cells present in the tumor include, without being limited to, anti-CD137 antibodies and anti-OX40 antibodies as described hereinabove.

Examples of antibodies inhibiting the growth or spreading of tumor cells include, without being limited to, anti-HER2 antibodies (such as trastuzumab).

According to one embodiment, the immunotherapy used for the treatment of a solid cancer with a combination comprising or consisting of metformin and cyclophosphamide as described hereinabove comprises or consists of an adoptive transfer of immune cells, in particular of T cells, as described above or of a checkpoint inhibitor therapy, in particular of a PD-1, PD-L1 and/or CTLA-4 inhibitor therapy, as described hereinabove.

The present invention relates to a combination comprising or consisting of metformin and cyclophosphamide as described hereinabove for use with an immunotherapy in the treatment of a solid cancer in a subject in need thereof, said combination being used as an adjuvant for the immunotherapy.

Thus, according to the present invention, the combination comprising or consisting of metformin and cyclophosphamide as described hereinabove is used as an adjuvant for an immunotherapy, in particular for a cancer immunotherapy. In other words, according to the present invention, the combination comprising or consisting of metformin and cyclophosphamide as described hereinabove potentiates an immunotherapy.

In one embodiment, potentiation of an immunotherapy in the presence of an adjuvant, in particular of a cancer immunotherapy, is defined by comparison with an immunotherapy, in particular a cancer immunotherapy, administered alone.

In one embodiment, said potentiation in the presence of an adjuvant of an immunotherapy, in particular of a cancer immunotherapy, is defined as at least one of the following, observed in the subject recipient of said immunotherapy:
increase of the number of tumor-infiltrated effector lymphocytes such as, for example, cytotoxic CD8+ T cells;
increase in the activation of tumor-infiltrated effector lymphocytes such as, for example, cytotoxic CD8+ T cells;
increase in the fitness of tumor-infiltrated effector lymphocytes such as, for example, cytotoxic CD8+ T cells, wherein fitness is assessed as the TCR-triggered signaling, proliferation and/or cytokine production by said effector lymphocytes and/or as the survival of said effector lymphocytes;
increase in the survival of tumor-infiltrated effector lymphocytes such as, for example, cytotoxic CD8+ T cells;
decrease of the number of tumor-infiltrated suppressive immune cells, such as suppressive myeloid cells (for example MDSCs and/or tumor-associated macrophages) and/or suppressive lymphocytes (for example T regulatory cells);
decrease in the activation of tumor-infiltrated suppressive immune cells, such as suppressive myeloid cells (for example MDSCs and/or tumor-associated macrophages) and/or suppressive lymphocytes (for example T regulatory cells);
decrease in the fitness of tumor-infiltrated suppressive immune cells, such as suppressive myeloid cells (for example MDSCs and/or tumor-associated macrophages) and/or suppressive lymphocytes (for example T regulatory cells), wherein fitness is assessed as the activation, proliferation and/or cytokine production by said suppressive immune cells, and/or as the survival of said suppressive immune cells;
decrease in the survival of tumor-infiltrated suppressive immune cells, such as suppressive myeloid cells (for example MDSCs and/or tumor-associated macrophages) and/or suppressive lymphocytes (for example T regulatory cells);
decrease in the tumor growth and/or in the tumor size; and/or
increase in survival.

The above-listed parameters are well-known to the person skilled in the art. Moreover, methods to determine the number, activation, fitness and/or survival of lymphocytes are commonly used in the field. Such methods include, for example, FACS analysis conducted on a tumor sample obtained from a subject (see for example Zhu et al., 2017, Nat Commun 8, 1404).

In one embodiment, the combination comprising or consisting of metformin and cyclophosphamide as described hereinabove for use according to the present invention improves effector T cell fitness in a hypoxic environment.

As used herein, a hypoxic environment refers to an environment deprived of oxygen.

In one embodiment, a hypoxic environment is defined as an environment with a level of oxygen ranging from about 0% to about 6%, preferably from about 0.2% to about 4.5%. In one embodiment, a hypoxic environment is defined as an environment with less than about 6%, 5.5%, 5%, 4.5%, 4%, 3.5%, 3%, 2.5%, 2%, 1.5%, 1%, or 0.5% oxygen.

Methods to determine whether an environment is hypoxic are well-known to the person skilled in the art. Such methods rely for example on the use of specific hypoxia markers such as anti-pimonidazole antibodies, anti-HIF-1a antibodies, anti-BNIP antibodies and in particular anti-BNIP3 antibodies, anti-PDK1 antibodies, anti-CAIX (carbonic anhydrase IX) antibodies and anti-GLUT1 antibodies; or of specific hypoxia reagents such as hypoxia green reagent for flow cytometry. Such methods can be carried out on a tumor sample obtained from a subject.

According to the present invention, metformin and cyclophosphamide in a combination of the invention are thus to be administered either simultaneously, separately or sequentially with respect to an immunotherapy for which they are used as an adjuvant.

Another object of the present invention is a method for treating a solid cancer in a subject in need thereof, said method comprising administering to the subject an immunotherapy and a combination comprising or consisting of metformin and cyclophosphamide as described hereinabove, wherein said combination comprising or consisting of metformin and cyclophosphamide is used as an adjuvant for the immunotherapy, thereby potentiating the immunotherapy. In one embodiment, the present invention relates to a method for treating a solid cancer in a subject in need thereof, said method comprising administering to the subject an immunotherapy and a combination of metformin and cyclophosphamide as described hereinabove, wherein said combination of metformin and cyclophosphamide is used as an adjuvant for the immunotherapy, thereby potentiating the immunotherapy.

According to one embodiment, said method for treating a solid cancer in a subject in need thereof comprises administering to the subject an immunotherapy and a combination comprising or consisting of metformin and cyclophosphamide as described hereinabove, wherein:
first, a therapeutically effective dose of cyclophosphamide is administered once to the subject;
then a therapeutically effective dose of metformin is administered to the subject prior to or concomitantly with an immunotherapy, preferably at least 1, 2, 3, 4, 5, 6, 7, 8, 9, or 10 days after the administration of a therapeutically effective dose of cyclophosphamide.

Another object of the present invention is a method for potentiating an immunotherapy in a subject treated for a solid cancer with an immunotherapy, said method comprising administering to the subject a combination comprising or consisting of metformin and cyclophosphamide as described hereinabove. In one embodiment, the present invention relates to a method for potentiating an immunotherapy in a subject treated for a solid cancer with an immunotherapy, said method comprising administering to the subject a combination of metformin and cyclophosphamide as described hereinabove.

According to one embodiment, said method for potentiating an immunotherapy in a subject in need thereof comprises administering to the subject an immunotherapy and a combination comprising or consisting of metformin and cyclophosphamide as described hereinabove, wherein:
first, a therapeutically effective dose of cyclophosphamide is administered once to the subject;
then a therapeutically effective dose of metformin is administered to the subject prior to or concomitantly with an immunotherapy, preferably at least 1, 2, 3, 4, 5, 6, 7, 8, 9, or 10 days, after the administration of a therapeutically effective dose of cyclophosphamide.

Another object of the present invention is the use of a combination comprising or consisting of metformin and cyclophosphamide as described hereinabove for the manufacture of a medicament for potentiating a cancer immunotherapy in a subject in need thereof. In one embodiment, the present invention relates to the use of a combination of metformin and cyclophosphamide as described hereinabove for the manufacture of a medicament for potentiating a cancer immunotherapy in a subject in need thereof.

Another object of the present invention is the use of a combination comprising or consisting of metformin and cyclophosphamide as described hereinabove for the manufacture of a medicament for the treatment of a solid cancer in a subject in need thereof, wherein said medicament is used as an adjuvant for an immunotherapy administered or to be administered to the subject. In one embodiment, the present invention relates to the use of a combination of metformin and cyclophosphamide as described hereinabove for the manufacture of a medicament for the treatment of a solid cancer in a subject in need thereof, wherein said medicament is used as an adjuvant for an immunotherapy administered or to be administered to the subject.

Another object of the present invention is a pharmaceutical combination comprising or consisting of metformin and cyclophosphamide as described hereinabove, and at least one pharmaceutically acceptable excipient, for use in the treatment of a solid cancer in a subject in need thereof, wherein said pharmaceutical combination is used as an adjuvant for an immunotherapy.

In one embodiment, the pharmaceutical combination for use in the treatment of a solid cancer in a subject in need thereof according to the invention comprises or consists of metformin and cyclophosphamide as described hereinabove, at least one pharmaceutically acceptable excipient, and an immunotherapy, such as, for example, a checkpoint inhibitor.

Another object of the present invention is a kit-of-parts comprising or consisting of a first part comprising a pharmaceutical composition comprising or consisting of metformin and at least one pharmaceutically acceptable excipient, and a second part comprising a pharmaceutical composition comprising or consisting of cyclophosphamide and at least one pharmaceutically acceptable excipient, for use in the treatment of a solid cancer in a subject in need thereof, wherein metformin and cyclophosphamide are used as an adjuvant for an immunotherapy.

In one embodiment, the kit-of parts for use in the treatment of a solid cancer in a subject in need thereof according to the invention comprises or consists of a first part comprising a pharmaceutical composition comprising or consisting of metformin and at least one pharmaceutically acceptable excipient, a second part comprising a pharmaceutical composition comprising or consisting of cyclophosphamide and at least one pharmaceutically acceptable excipient, and a third part comprising or consisting of an immunotherapy, such as, for example, a checkpoint inhibitor.

Pharmaceutically acceptable excipients that may be used in the pharmaceutical combination or composition of the invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances (for example sodium carboxymethylcellulose), polyethylene glycol, polyacrylates, waxes, polyethylene-polyoxypropylene-block polymers, polyethylene glycol and wool fat.

Another object of the invention is a medicament comprising or consisting of a combination of metformin and cyclophosphamide as described hereinabove, or a pharmaceutical combination as described hereinabove, or a kit-of parts as described hereinabove, for use in the treatment of a solid cancer in a subject in need thereof, wherein said medicament is used as an adjuvant for an immunotherapy.

As mentioned hereinabove, according to the present invention, metformin and cyclophosphamide in a combination of the invention are to be administered either simultaneously, separately or sequentially with respect to each other.

Moreover, as mentioned hereinabove, metformin and cyclophosphamide in a combination of the invention are to be administered either simultaneously, separately or sequentially with respect to the immunotherapy for which they are used as an adjuvant.

According to one embodiment, metformin and cyclophosphamide, the combination or pharmaceutical combination thereof, medicament or kit-of-parts according to the invention will be formulated for administration to the subject. Metformin and cyclophosphamide, the combination or pharmaceutical combination thereof, or medicament according to the invention may be administered orally, parenterally, topically, by inhalation spray, rectally, nasally, buccally, vaginally or via an implanted reservoir.

In one embodiment, metformin as described hereinabove is in an adapted form for an oral administration. Thus, in one embodiment, metformin is to be administered orally to the subject, for example as a powder, a tablet, a capsule or as a tablet formulated for extended or sustained release. In another embodiment, metformin as described hereinabove is in an adapted form for an injection. Thus, in one, metformin as described hereinabove is to be injected to the subject, by intravenous, intramuscular, intraperitoneal, intrapleural, subcutaneous, transdermal injection or infusion.

In one embodiment, cyclophosphamide as described hereinabove is in an adapted form for an oral administration. Thus, in one embodiment, cyclophosphamide as described hereinabove is to be administered orally to the subject, for example as a capsule or as a tablet. In another embodiment, cyclophosphamide as described hereinabove is in an adapted form for an injection. Thus, in another embodiment, cyclophosphamide as described hereinabove is to be injected to the subject, by intravenous, intramuscular, intraperitoneal, intrapleural, subcutaneous, transdermal injection or infusion, preferably by intravenous injection.

In one embodiment, the combination, pharmaceutical combination, medicament or kit-of-parts according to the invention is in a form adapted for oral administration. In other words, the combination, pharmaceutical combination, medicament or kit-of-parts according to the invention comprises metformin and cyclophosphamide which are both in a form adapted for oral administration. Thus, in one embodiment, the combination, pharmaceutical combination, medicament or kit-of-parts according to the invention comprises metformin and cyclophosphamide which are to be administered orally to the subject.

Examples of forms adapted for oral administration include, without being limited to, liquid, paste or solid compositions, and more particularly tablets, tablets formulated for extended or sustained release, capsules, pills, dragees, liquids, gels, syrups, slurries, suspensions, and the like.

In one embodiment, the combination, pharmaceutical combination, medicament or kit-of-parts according to the invention is in a form adapted for parenteral administration. In other words, the combination, pharmaceutical combination, medicament or kit-of-parts according to the invention comprises metformin and cyclophosphamide which are both in a form adapted for parenteral administration. Thus, in one embodiment, the combination, pharmaceutical combination, medicament or kit-of-parts according to the invention comprises metformin and cyclophosphamide which are to be administered parenterally.

In one embodiment, the combination, pharmaceutical combination, medicament or kit-of-parts according to the invention is in a form adapted for injection, such as, for example, for intravenous, subcutaneous, intramuscular, intradermal, transdermal injection or infusion. In other words, the combination, pharmaceutical combination, medicament or kit-of-parts according to the invention comprises metformin and cyclophosphamide which are both in a form adapted for injection, such as, for example, for intravenous, intramuscular, intraperitoneal, intrapleural, subcutaneous, transdermal injection or infusion. Thus, in one embodiment, the combination, pharmaceutical combination, medicament or kit-of-parts according to the invention comprises metformin and cyclophosphamide which are to be administered by injection to the subject, such as, for example, by intravenous, intramuscular, intraperitoneal, intrapleural, subcutaneous, transdermal injection or infusion.

Sterile injectable forms of metformin and cyclophosphamide, the combination or pharmaceutical combination thereof, or medicament according to the invention may be a solution or an aqueous or oleaginous suspension. These suspensions may be formulated according to techniques known in the art using suitable dispersing or wetting agents and suspending agents. The sterile injectable preparation may also be a sterile injectable solution or suspension in a non-toxic pharmaceutically acceptable diluent or solvent. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

In one embodiment, the combination, pharmaceutical combination, medicament or kit-of-parts according to the invention comprises metformin that is in a form adapted for oral administration and cyclophosphamide that is in a form adapted for injection, such as, for example, for intravenous, intramuscular, intraperitoneal, intrapleural, subcutaneous, transdermal injection or infusion. Thus, in one embodiment, the combination, pharmaceutical combination, medicament or kit-of-parts according to the invention comprises metformin that is to be administered orally and cyclophosphamide that is to be administered by injection to the subject, such as, for example, by intravenous, intramuscular, intraperitoneal, intrapleural, subcutaneous, transdermal injection or infusion.

In one embodiment, the combination, pharmaceutical combination, medicament or kit-of-parts according to the invention comprises metformin that is in a form adapted for injection, such as, for example, for intravenous, intramuscular, intraperitoneal, intrapleural, subcutaneous, transdermal injection or infusion and cyclophosphamide that is in a form adapted for oral administration. Thus, in one embodiment, the combination, pharmaceutical combination, medicament or kit-of-parts according to the invention comprises metformin that is to be administered by injection to the subject, such as, for example, by intravenous, intramuscular, intraperitoneal, intrapleural, subcutaneous, transdermal injection or infusion and cyclophosphamide that is to be administered orally.

In another embodiment, the combination, pharmaceutical combination, medicament or kit-of-parts according to the invention is in a form adapted for topical administration. In other words, the combination, pharmaceutical combination, medicament or kit-of-parts according to the invention comprises metformin and cyclophosphamide which are both in a form adapted for topical administration. Thus, in one embodiment, the combination, pharmaceutical combination, medicament or kit-of-parts according to the invention comprises metformin and cyclophosphamide which are to be administered topically to the subject.

Examples of forms adapted for topical administration include, without being limited to, liquid, paste or solid compositions, and more particularly aqueous solutions, drops, dispersions, sprays, microcapsules, micro- or nanoparticles, polymeric patch, or controlled-release patch, and the like.

In another embodiment, the combination, pharmaceutical combination, medicament or kit-of-parts according to the invention is in a form adapted for rectal administration. In other words, the combination, pharmaceutical combination, medicament or kit-of-parts according to the invention comprises metformin and cyclophosphamide which are both in a form adapted for rectal administration. Thus, in one embodiment, the combination, pharmaceutical combination, medicament or kit-of-parts according to the invention comprises metformin and cyclophosphamide which are to be administered rectally.

Examples of forms adapted for rectal administration include, without being limited to, suppository, micro enemas, enemas, gel, rectal foam, cream, ointment, and the like.

In one embodiment, cyclophosphamide is to be administered prior to the immunotherapy.

In one embodiment, cyclophosphamide is to be administered prior to the immunotherapy, once, twice, three times or more.

In one embodiment, cyclophosphamide is to be administered prior to the immunotherapy, once a day for 1, 2, 3, 4, 5 or more days.

In one embodiment, cyclophosphamide is to be administered once prior to the immunotherapy.

In one embodiment, cyclophosphamide is to be administered at least 1, 2, 3, 4, 5, 6, 7, 8, 9, or 10 days before the immunotherapy. In one embodiment, the immunotherapy is an adoptive cell therapy and cyclophosphamide is to be administered at least 1, 2, 3, 4, 5, 6, 7, 8, 9, or 10 days, preferably at least 1 day, before the immune cells, in particular the T cells, as described above are transferred. In another embodiment, the immunotherapy is a checkpoint inhibitor therapy and cyclophosphamide is to be administered at least 1, 2, 3, 4, 5, 6, 7, 8, 9, or 10 days, preferably at least 1 day, before the checkpoint inhibitor as described hereinabove is administered.

In one embodiment, metformin is to be administered after cyclophosphamide. In one embodiment, metformin is to be administered at least 1, 2, 3, 4, 5, 6, 7, 8, or 10 days after cyclophosphamide

In one embodiment, metformin is to be administered prior to or concomitantly with the immunotherapy. In one embodiment, the immunotherapy is an adoptive cell therapy and metformin is to be administered prior to the day or on the same day that the immune cells, in particular the T cells, as described hereinabove are transferred. In another embodiment, the immunotherapy is a checkpoint inhibitor and metformin is to be administered prior to the day or on the same day that the checkpoint inhibitor as described hereinabove is administered.

In one embodiment, metformin is to be administered after cyclophosphamide and prior to or concomitantly with the immunotherapy. Thus, in one embodiment, cyclophosphamide is to be administered prior to the immunotherapy, as described hereinabove, and prior to metformin, with metformin to be administered prior to or concomitantly with said immunotherapy, as described hereinabove.

In one embodiment, metformin is to be administered prior to or concomitantly with the immunotherapy as described hereinabove and continuously thereafter.

In one embodiment, metformin is to be administered prior to or concomitantly with the immunotherapy and subsequently for at least 1, 2, 3, 4, 5, 6, 7, 8, 9, or 10 days thereafter. In another embodiment, metformin is to be administered prior to or concomitantly with the immunotherapy and subsequently for at least 1, 2, 3, 4, 5, 6, 7, 8, 9, or 10 weeks thereafter. In another embodiment, metformin is to be administered prior to or concomitantly with the immunotherapy and subsequently for at least 1, 2, 3, 4, 5, 6, 7, 8, 9, or 10 months thereafter.

In one embodiment, the immunotherapy is an adoptive cell therapy and metformin is to be administered prior to or concomitantly with said adoptive cell therapy and continuously thereafter. In one embodiment, the immunotherapy is an adoptive cell therapy and metformin is to be administered prior to or concomitantly with said adoptive cell therapy and subsequently for at least 1, 2, 3, 4, 5, 6, 7, 8, 9, or 10 weeks thereafter.

In one embodiment, the immunotherapy is a checkpoint inhibitor therapy and metformin is to be administered prior to or concomitantly with said checkpoint inhibitor therapy and continuously thereafter. In one embodiment, the immunotherapy is a checkpoint inhibitor therapy and metformin is to be administered prior to or concomitantly with said checkpoint inhibitor therapy and subsequently for at least 1, 2, 3, 4, 5, 6, 7, 8, 9, or 10 weeks thereafter.

In one embodiment of the invention, cyclophosphamide is to be administered prior to the immunotherapy, preferably at least 1 day before the immunotherapy, and prior to metformin;

and metformin is to be administered prior to or concomitantly with the immunotherapy, and continuously thereafter.

According to one embodiment, a therapeutically effective dose of metformin as described hereinabove is to be administered in combination with a therapeutically effective dose of cyclophosphamide as described hereinabove for use in the treatment of a solid cancer in a subject in need thereof, wherein said metformin in combination with cyclophosphamide is used as an adjuvant for an immunotherapy. Thus, in one embodiment, the combination, pharmaceutical combination, medicament or kit-of-parts according to the invention as described hereinabove comprises a therapeutically effective dose of metformin as described hereinabove and a therapeutically effective dose of cyclophosphamide as described hereinabove.

It will be understood that the total daily usage of metformin and the total daily usage of cyclophosphamide in combination according to the invention will be decided by the attending physician within the scope of sound medical judgment. The specific dose for any particular subject will depend upon a variety of factors such as the solid cancer to be treated; the age, body weight, general health, sex and diet of the patient; and like factors well-known in the medical arts.

In one embodiment, the subject is a mammal, preferably a human, and said therapeutically effective dose of metformin is a dose ranging from about 0.15 mg per kilo body weight per day (mg/kg/day) to about 150 mg/kg/day, preferably from about 1.5 mg/kg/day to about 100 mg/kg/day, and more preferably from about 7 mg/kg/day to about 75 mg/kg/day.

In one embodiment, the subject is a mammal, preferably a human, and said therapeutically effective dose of metformin is a daily dose ranging from about 10 mg to about 10000 mg, preferably from about 100 mg to about 7000 mg, and more preferably from about 500 mg to about 5000 mg.

In one embodiment, the subject is a mammal, preferably a human, and said therapeutically effective dose of metformin is a dose of at least about 7, 7.5, 8.5, 9, 10, 10.5, 11, 12, 12.5, 13.5, 14, 17.5, 21, 25, 28, 32, 35, 39, 42.5, 46, 50, 53, 57, 60.5, 64, 67.5, or 71 mg/kg/day.

In one embodiment, the subject is a mammal, preferably a human, and said therapeutically effective dose of metformin is a daily dose of at least about 500, 550, 600, 650, 700, 750, 800, 850, 900, 950, 1000, 1250, 1500, 1750, 2000, 2250, 2500, 2750, 3000, 3250, 3500, 3750, 4000, 4250, 4500, 4750 or 5000 mg.

In one embodiment, the subject is a mammal, preferably a human, and said therapeutically effective dose of metformin is a dose of about 7, 10, 12, 14, 17.5, 21, 25, 28, 32 or 35 mg/kg/day.

In one embodiment, the subject is a mammal, preferably a human, and said therapeutically effective dose of metformin is a daily dose of about 500, 750, 850, 1000, 1250, 1500, 1750, 2000, 2250, or 2500 mg.

In one embodiment, the subject is a mammal, preferably a human, and said therapeutically effective dose of metformin is a daily dose to be administered in one, two, three or more take(s).

In one embodiment, the subject is a mammal, preferably a human, and said therapeutically effective dose of metformin is a daily dose to be administered in one or two take(s).

In one embodiment, the subject is a mammal, preferably a human, and said therapeutically effective dose of cyclophosphamide is a dose ranging from about 0.15 mg per kilo body weight per day (mg/kg/day) to about 150 mg/kg/day, preferably from about 0.7 mg/kg/day to about 100 mg/kg/day, and more preferably from about 2 mg/kg/day to about 75 mg/kg/day.

In one embodiment, the subject is a mammal, preferably a human, and said therapeutically effective dose of cyclophosphamide is a daily dose ranging from about 5.5 mg/m² body surface area (hereafter referred to as mg/m²) to about 5500 mg/m², preferably from about 25 mg/m² to about 3850 mg/m², and more preferably from about 200 mg/m² to about 3000 mg/m².

In one embodiment, the subject is a mammal, preferably a human, and said therapeutically effective dose of cyclophosphamide is a daily dose ranging from about 10 mg to about 10000 mg, preferably from about 50 mg to about 7000 mg, and more preferably from about 150 mg to about 5000 mg.

In one embodiment, the subject is a mammal, preferably a human, and said therapeutically effective dose of cyclophosphamide is a dose of at least about 2, 2.5, 7, 10, 14, 21, 28, 35, 42.5, 50, 57, 60, or 64 mg/kg/day.

In one embodiment, the subject is a mammal, preferably a human, and said therapeutically effective dose of cyclophosphamide is a daily dose of at least about 150, 200, 500, 700, 1000, 1500, 2000, 2500, 3000, 3500, 4000, 4200 or 4500 mg.

In one embodiment, the subject is a mammal, preferably a human, and said therapeutically effective dose of cyclophosphamide is a dose of about 7, 14, 21, 28, 35, 42.5, 50, 57, 60, or 64 mg/kg/day.

In one embodiment, the subject is a mammal, preferably a human, and said therapeutically effective dose of cyclophosphamide is a daily dose of about 500, 1000, 1500, 2000, 2500, 3000, 3500, 4000, 4200 or 4500 mg.

In one embodiment, the subject is a mammal, preferably a human, and said therapeutically effective dose of cyclophosphamide is a daily dose to be administered in one take or in one injection.

According to the present invention, the combination comprising or consisting of metformin and cyclophosphamide as described hereinabove is for use with an immunotherapy in the treatment of a solid cancer in a subject in need thereof.

As used herein, the term “solid cancer” encompasses any cancer (also referred to as malignancy) that forms a discrete tumor mass, as opposed to cancers (or malignancies) that diffusely infiltrate a tissue without forming a mass.

Examples of solid cancers include, without being limited to, adrenocortical carcinoma, anal cancer, basal cell carcinoma, bile duct cancer, bladder cancer, bone cancer, brain cancer such as glioblastoma or central nervous system (CNS) tumors, breast cancer (such as triple negative breast cancer and inflammatory breast cancer), cervical cancer, uterine cancer, endometrial cancer, colorectal cancer (CRC) such as colon carcinoma, esophageal cancer, eye cancer such as retinoblastoma, gallbladder cancer, gastric cancer (also referred to as stomach cancer), gastrointestinal carcinoma, gastrointestinal stromal tumor (GIST), head and neck cancer (such as for example laryngeal cancer, oropharyngeal cancer, nasopharyngeal carcinoma, or throat cancer), liver cancer such as hepatocellular carcinoma (HCC), Hodgkin's lymphoma, Kaposi sarcoma, mastocytosis, myelofibrosis, lung cancer (such as lung carcinoma, non-small-cell lung carcinoma (NSCLC), and small cell lung cancer), pleural mesothelioma, melanoma such as uveal melanoma, neuroendocrine tumors, neuroblastoma, ovarian cancer, primary peritoneal cancer, pancreatic cancer, parathyroid cancer, penile cancer, pituitary adenoma, prostate cancer such as castrate metastatic prostate cancer, rectal cancer, kidney cancer such as renal cell carcinoma (RCC), skin cancer other than melanoma such as Merkel cell skin cancer, small intestine cancer, sarcoma such as soft tissue sarcoma, squamous-cell carcinoma, testicular cancer, thyroid cancer, and urethral cancer.

In one embodiment, the solid cancer to be treated according to the invention is selected from the group comprising melanoma, Merkel cell skin cancer, Hodgkin's Lymphoma, lung cancer, head and neck cancer, bladder cancer, kidney cancer, cervical cancer, pancreatic cancer, prostate cancer, breast cancer, gastric cancer, and glioblastoma.

In one embodiment, the solid cancer to be treated according to the invention is selected from the group comprising melanoma, Merkel cell skin cancer, Hodgkin's Lymphoma, lung cancer, head and neck cancer, bladder cancer, colon cancer, kidney cancer, cervical cancer, pancreatic cancer, prostate cancer, breast cancer, gastric cancer, and glioblastoma.

In one embodiment, the solid cancer to be treated according to the invention is selected from the group comprising or consisting of melanoma, Merkel cell skin cancer, Hodgkin's Lymphoma, lung cancer, head and neck cancer, bladder cancer, and kidney cancer.

In one embodiment, the solid cancer to be treated according to the invention is not a lymphoid cancer. Thus, in one embodiment, the solid cancer to be treated according to the invention is not lymphoma (including non-Hodgkin lymphoma and Hodgkin lymphoma).

In one embodiment, the solid cancer to be treated according to the invention is a metastatic solid cancer, i.e., a solid cancer wherein at least one metastatic tumor is observed in addition to the primary tumor.

In one embodiment, the solid cancer to be treated according to the invention is selected from the group comprising or consisting of melanoma, such as uveal melanoma, pancreatic cancer, lung cancer such as lung carcinoma or non-small cell lung cancer, pleural mesothelioma, ovarian cancer, primary peritoneal cancer, prostate cancer, such as castrate metastatic prostate cancer, gastrointestinal carcinoma, breast cancer, liver cancer such as hepatocellular carcinoma, sarcoma, and central nervous system (CNS) tumors.

In one embodiment, the solid cancer to be treated according to the invention is selected from the group comprising or consisting of melanoma, Merkel cell skin cancer, lung cancer, head and neck cancer, bladder cancer, colon cancer, kidney cancer, cervical cancer, pancreatic cancer, prostate cancer, breast cancer, gastric cancer, and glioblastoma.

In one embodiment, the solid cancer to be treated according to the invention is selected from the group comprising or consisting of melanoma, Merkel cell skin cancer, lung cancer, head and neck cancer, bladder cancer, and kidney cancer.

In one embodiment, the solid cancer to be treated according to the invention is selected from the group comprising or consisting of cervical cancer, pancreatic cancer, prostate cancer, breast cancer, gastric cancer, and glioblastoma.

In one embodiment, the solid cancer to be treated according to the invention is selected from the group comprising or consisting of melanoma, colorectal cancer such as colon carcinoma, lung cancer, head and neck cancer, and bladder cancer.

In one embodiment, the solid cancer to be treated according to the invention is selected from the group comprising or consisting of melanoma, colorectal cancer such as colon carcinoma and lung cancer such as lung carcinoma, for example Lewis lung carcinoma.

In one embodiment, the solid cancer to be treated according to the invention is melanoma or colon carcinoma.

In one embodiment, the solid cancer to be treated according to the invention is melanoma.

### BRIEF DESCRIPTION OF THE DRAWINGS

FIG. 1 is a group of histograms showing the infiltration of TCRP1A CD8⁺ T cells in tumor (A) or in hypoxic (pimonidazole-positive) tumor regions (B) of TiRP mice, 4 days after adoptive cell transfer (ACT). The mice were transferred 10⁷ of activated TCRP1A CD8⁺ T cells and were treated as indicated with vehicle (H2O) or metformin (0.5 mg/ml) from the moment of ACT. Infiltration of TCRP1A CD8⁺ T cells (A) was assessed as the percentage of P1A tetramer⁺ cells among alive tumor infiltrating CD8⁺ cells determined using FACS analysis. Hypoxic TCRP1A CD8⁺ T cells (B) were quantified as pimonidazole⁺ cells among P1A tetramer⁺ tumor infiltrating CD8⁺ alive cells using FACS analysis. Results are expressed as mean values±s.e.m; *=p<0.05.

FIG. 2 is a group of histograms showing tumor infiltrating regulatory T cells (Treg cells) (A) or tumor infiltrating polymorphonuclear myeloid-derived suppressor cells (PMN-MDSCs) (B) in TiRP mice, 4 days after adoptive cell transfer (ACT). The mice received a single injection of cyclophosphamide (CTX, 100 mg/kg) or vehicle (PBS) as indicated when the tumor size was around 500 mm³, one day before the ACT of 10⁷ of activated TCRP1A CD8⁺ T cells. The mice were sacrificed 4 days later. PMN-MDSCs were distinguished from neutrophils by a density separation through a Lymphoprep gradient. Infiltration of Treg was assessed as the percentage of FoxP3⁺ CD25⁺ among live CD4⁺ cells determined using FACS analysis. Infiltration of PMN-MDSCs was assessed as the percentage of Ly6G^{hi}Ly6C^{low} CD11b⁺ among live cells determined using FACS analysis. Results are expressed as mean values±s.e.m; *=p<0.05.

FIG. 3 is a group of histograms showing the infiltration (A) and apoptosis (B) of TCRP1A CD8⁺ T cells in tumors of TiRP mice, 14 days after adoptive cell transfer (ACT). The mice received a single injection of cyclophosphamide (CTX, 100 mg/kg) or vehicle (PBS) as indicated when the tumor size was around 500 mm³, one day before the ACT of 10⁷ of activated TCRP1A CD8⁺ T cells. From the day of the ACT until the mice were sacrificed, metformin was added or not (vehicle) in the mice drinking water at a concentration of 0.5 mg/ml. The mice were sacrificed 14 days later. Infiltration of TCRP1A CD8⁺ T cells was assessed as the percentage of P1A tetramer⁺ cells among alive tumor infiltrating CD8⁺ cells determined using FACS analysis. Apoptosis of TCRP1A CD8⁺ T cells was assessed with annexin V staining Results are expressed as mean values±s.e.m; *=p<0.05.

FIG. 4 is a group of histograms showing the infiltration (A) and apoptosis (B) of TCRP1A CD8⁺ T cells in hypoxic (pimonidazole-positive) tumor regions of TiRP mice, 14 days after adoptive cell transfer (ACT). The mice received a single injection of cyclophosphamide (CTX, 100 mg/kg) or vehicle (PBS) as indicated when the tumor size was around 500 mm³, one day before the ACT of 10⁷ of activated TCRP1A CD8⁺ T cells. From the day of the ACT until the mice were sacrificed, metformin was added or not (vehicle) in the mice drinking water at a concentration of 0.5 mg/ml. The mice were sacrificed 14 days later. Infiltration of TCRP1A CD8⁺ T cells in hypoxic tumor regions was assessed as the percentage of P1A tetramer+CD8⁺ cells among alive pimonidazole⁺ cells determined using FACS analysis. Apoptosis of pimonidazole⁺ TCRP1A CD8⁺ T cells was assessed with annexin V staining Results are expressed as mean values±s.e.m; *=p<0.05.

FIG. 5 is a group of graphs showing tumor growth (A) and end-stage tumor weight (B) in TiRP mice after adoptive cell transfer (ACT). The mice received a single injection of cyclophosphamide (CTX, 100 mg/kg) or vehicle (PBS) as indicated when the tumor size was around 500 mm³, one day before the ACT of 10⁷ of activated TCRP1A CD8⁺ T cells. From the day of the ACT until the mice were sacrificed, metformin was added or not (vehicle) in the mice drinking water at a concentration of 0.5 mg/ml. Tumor growth was assessed at the indicated time after ACT. End-stage tumor weight was assessed 14 days after ACT. Results are expressed as mean values±s.e.m; *=p<0.05. (A) Circles=control condition (PBS+vehicle); triangles=cyclophosphamide alone (CTX+vehicle); squares=metformin alone (PBS+metformin); inverted triangles=cyclophosphamide and metformin (CTX+metformin).

FIG. 6 is a group of graphs showing tumor growth (A) and survival (B) of TiRP mice after adoptive cell transfer (ACT). The mice received a single injection of cyclophosphamide (CTX, 100 mg/kg) or vehicle (PBS) as indicated when the tumor size was around 500 mm³, one day before the ACT of 10⁷ of activated TCRP1A CD8⁺ T cells. From the day of the ACT, metformin was added or not (vehicle) in the mice drinking water at a concentration of 0.5 mg/ml until the mice were sacrificed or until the end of the experiment. Tumor growth was assessed at the indicated time after ACT, up to 22 days after ACT or when any humane endpoint was reached. (A) Results are expressed as mean values±s.e.m; *=p<0.05, metformin vs. vehicle; # =p<0.05, CTX vs. PBS. (B) Survival is expressed as a Kaplan-Meier survival curve; *=p<0.05, metformin vs. vehicle; # =p<0.05, CTX vs. PBS. Diamonds (A) or dashed line (B)=control condition with adoptive cell transfer (ACT) (PBS+vehicle+ACT); squares (A) or continuous line (B)=cyclophosphamide alone with ACT (CTX+vehicle+ACT); circles (A) or dashed and dotted line (B)=metformin alone with ACT (PBS +metformin+ACT); triangles (A) or dotted line (B)=cyclophosphamide and metformin with ACT (CTX+metformin+ACT).

FIG. 7 is a graph showing tumor growth in TiRP mice following adoptive cell transfer (ACT) (continuous lines) or not (dotted lines). The mice received a single injection of cyclophosphamide (CTX, 100 mg/kg) or vehicle (PBS) as indicated when the tumor size was around 500 mm3, followed or not with the ACT of 10⁷ of activated TCRP1A CD8+ T cells 24 hours later. From the day of the ACT until the mice were sacrificed, metformin was added or not (vehicle) in the mice drinking water at a concentration of 0.5 mg/ml. Tumor growth was assessed at the indicated time after ACT, up to 22 days after ACT or when any humane endpoint was reached. Results are expressed as mean values±s.e.m; *=p<0.05, metformin vs. vehicle; # =p<0.05, CTX vs. PBS; S=p<0.05, ACT vs. no ACT. Circles=control condition; squares=cyclophosphamide alone; diamonds=metformin alone; triangles=cyclophosphamide and metformin. Continuous lines=with adoptive cell transfer (ACT); dotted lines=without ACT.

FIG. 8 is a group of graphs showing tumor growth in B6 mice treated with cyclophosphamide (CTX) and/or metformin in combination with anti-PD-1 antibody or isotype control. The mice were subcutaneously injected with B16F1 melanoma cells. One week after said injection, the mice were randomized according to the tumor size and injected with CTX (100 mg/kg) or control (PBS) (day −1). Starting from the following day (day 0) and until the end of the experiment, metformin was added or not (vehicle) in the mice drinking water at a concentration of 0.5 mg/ml. In combination, mice received an intraperitoneal injection of 100 μg of anti-PD-1 antibody (clone RMP1-14) or RatIgG2a isotype (clone 2A3) on day 0, day 3 and day 7. Tumor growth was assessed at the indicated time points after CTX/PBS treatment. Results are expressed as mean values±s.e.m; *=p<0.05. (A) Comparison of all the conditions tested; (B) Comparison of the combinations CTX+metformin and isotype control vs. CTX+metformin and anti-PD-1 antibody; (C) Comparison of the combinations PBS+metformin and anti-PD-1 antibody vs. CTX+metformin and anti-PD-1 antibody; and (D) Comparison of the combinations CTX+vehicle and anti-PD-1 antibody vs. CTX+metformin and anti-PD-1 antibody.

FIG. 9 is a group of graphs showing tumor growth in B6 mice treated with cyclophosphamide (CTX) and/or metformin in combination with anti-PD-1 antibody or isotype control. The mice were subcutaneously injected with MC38 colon carcinoma cells. When the average tumor size was between 90 and 120 mm³, the mice were randomized and injected with CTX (100 mg/kg) or control (PBS) (day −1). Starting from the following (day 0) and until the end of the experiment, metformin was added or not (vehicle) in the mice drinking water at a concentration of 0.5 mg/ml. In combination, mice received an intraperitoneal injection of 100 μg of anti-PD-1 antibody (clone RMP1-14) or RatIgG2a isotype (clone 2A3) on day 0, day 3, day 6 and day 9. Tumor growth was assessed at the indicated time points after CTX/PBS treatment. Each graph shows individual tumor growth in all the different conditions tested: (A) PBS+vehicle+anti-PD-1; (B) PBS+metformin+anti-PD-1; (C) CTX+vehicle+anti-PD-1; (D) CTX +metformin+anti-PD-1; (E) PBS+vehicle+isotype; (F) PBS+metformin+isotype; (G) CTX+vehicle+isotype; (H) CTX+metformin+isotype. The number of mice that had rejected the tumor 16 days after the beginning of the treatment is indicated for each condition.

### EXAMPLES

The present invention is further illustrated by the following examples.

### Example 1:

Materials and Methods

Material

Mice

TiRP mice have been created by crossing Ink4a/Arf^{flox/flox} mice with mice carrying a transgenic construct controlled by the tyrosinase promoter and driving the expression of H-Ras^{12V} and Trap 1 a which encodes a MAGE-type tumor antigen P1A; the promoter is separated from the coding region by a stop cassette made of a floxed self-deleting CreER (Huijbers et al., 2006, Cancer Res 66, 3278-3286). Those mice were backcrossed to a B10.D2 background and bred to homozygosity. TCRP1A mice heterozygous for the H-2Ld/P1A35-43-specific TCR transgene were kept on the B10.D2;Rag1^{-/-} background (Shanker et al., 2004, J Immunol 172, 5069-5077).

Gender and age matched CD57BL/6 wild-type mice were used for the experiments with checkpoint inhibitors.

All mice used in this study were produced under specific pathogen free (SPF) conditions at the animal facility of the Ludwig Institute for Cancer Research. All the rules concerning animal welfare have been respected according to the 2010/63/EU Directive. All procedures were performed with the approval of the local Animal Ethical Committee, with reference 2015/UCL/MD/15.

Methods

Tumor Induction and Mice Treatments

A fresh solution of 4OH-Tamoxifen was prepared by dissolving 4OH-Tamoxifen (Imaginechem) in 100% ethanol and mineral oil (ratio 1:9) followed by 30-min sonication, and injected subcutaneously (2 mg/200 μl per mouse) in the neck area of gender-matched 7 weeks old TiRP mice. Tumor appearance was monitored daily and tumors were measured three times/week. Tumor volume (in mm³) was calculated by the following formula: Volume=width²×length×π/6. Tumor-bearing TiRP mice were randomized based on the tumor size when average volume was 500 mm³ and treated as indicated. For cyclophosphamide (CTX, Sigma C7397) treatment, mice received an intra-peritoneal injection of CTX at 100 mg/kg or vehicle (PBS) 24 h before adoptive cell transfer. For metformin treatment, metformin (Enzo, ALX-270-432) was dissolved in mice drinking water at a concentration of 0.5 mg/ml and administered to the mice from the moment of adoptive cell transfer until the end of the experiment; the solution was replaced three times/week.

For the experiments with checkpoint inhibitors, gender matched, 7-9 weeks old CD57BL/6 wild-type mice were subcutaneously injected with 1 million of B16F1 or MC38 tumor cells. Mice were randomized based on the tumor size one week after the injection of tumor cells (B16F1 melanoma model) or when the average tumor size was between 90 and 120 mm³ (MC38 colon carcinoma model). Tumor-bearing mice were then treated with CTX or PBS as described above. One day after, metformin was prepared as mentioned above and added to the mice drinking water until the end of the experiment. A dose of 100 μg/mouse of anti-PD-1 antibody (clone RMP1-14, Bio-X-Cell) or RatIgG2a isotype (clone 2A3, Bio-X-Cell) was injected intraperitoneally three times (B16F1 model) or four times (MC38 model) every three or four days starting from the day after CTX or PBS treatment.

Adoptive Cell Rransfer with TCRP1A CD8⁺ T Cells

For the adoptive cell transfer (ACT), P1A-specific (TCRP1A) CD8⁺ T cells were isolated from spleens and lymph nodes of TCRP1A mice using anti-mouse CD8α (Ly-2) MicroBeads (Miltenyi Biotec), and stimulated in vitro by co-culture with irradiated (10.000 rads) L1210.P1A.B7-1 cells (Gajewski et al., 1995, J Immunol 154, 5637-5648) at 1:2 ratio (0.5×10⁵ CD8⁺ T cells and 10⁵ L1210.P1A.B7-1 cells per well in 48-well plates) in IMDM (GIBCO) containing 10% fetal bovine serum supplemented with L-arginine (0.55 mM, Merck), L-asparagine (0.24 mM, Merck), glutamine (1.5 mM, Merck), betamercaptoethanol (50 μM, Sigma), 50 Uml⁻¹ penicillin and 50 mg m1⁻¹ streptomycin (Life Technologies). Four days later, TCRP1A CD8⁺ T cells were purified on a Lymphoprep gradient (StemCell) and 10⁷ living cells were injected intravenously in 200 μl PBS in TiRP-tumor bearing mice.

FACS Analysis

For hypoxia detection, mice received an intraperitoneal injection of pimonidazole hydrochloride at 60 mg/kg (Hypoxyprobe) 45 minutes before tumor harvesting. Tumor single cell suspension was prepared using gentleMACS Dissociator and incubating the tissue in RMPI containing 0.04% collagenase I, 0.04% collagenase II and 0.05% dispase (all from GIBCO) for 1 hour at 37° C. After passing the cell suspension through a 70 μm- and 40 μm-cell strainer, red blood cell lysis was performed (eBioscience, 00-4300-54). Tumor cell preparation was stained with the Viability dye eFluor780 (eBioscience, 1:1000) to identify dead cells during the FACS analysis, and then incubated with Mouse BD Fc Block (BD, clone 2.4G2, 1:100) in PBS supplemented with 2% FBS and 2 mM EDTA. TCRP1A CD8⁺ T cells were labeled by using H-2L^{d} PIA tetramer-PE (1 μM, produced in-house at the Ludwig Institute for Cancer Research) and anti-CD8α BV-421 (BioLegend, 53-6.7, 1:200); for apoptosis quantification, Annexin V APC (BioLegend, 1:20) was used. Hypoxic cells were identified with the anti-pimonidazole antibody (Hypoxyprobe, HP-FITC-Mab 4.3.11.3, 1:100) after cell fixation and permeabilization according to manufacturer's instructions (eBioscience Transcription Factor Staining Buffer Set). Treg cells were identified by anti-CD4 FITC (BioLegend, GK1.5, 1:200) and anti-CD25 BV-421 (BioLegend, PC61, 1:200) extracellular staining, followed by anti-FoxP3 APC (eBioscience, FJK-16s, 1:100) intracellular staining after cell fixation and permeabilization as above. For PMN-MDSC quantification, tumor cell suspension was applied onto a Lymphoprep gradient, to separate high-density cells (neutrophils) from low-density or mononuclear cells; the low-density fraction was stained for CD11b BV421 (BioLegend, M1/70, 1:200), Ly6C PerCP (BioLegend, HK1.4, 1:200), Ly6G PE (BioLegend, 1A8, 1:200) to identify PMN-MDSCs. Stained samples were acquired by FACS Fortessa (BD Bioscience) and analyzed with FlowJo, LLC v.10.1.

Statistics

Data entry and all analyses were performed in a blinded fashion. All statistical analyses were performed using GraphPad Prism software. Statistical significance was calculated by two-tailed unpaired t-test on two experimental conditions or two-way analysis of variance (ANOVA) when repeated measures were compared, with p<0.05 considered statistically significant. Independent experiments were pooled and analyzed together whenever possible. All graphs show mean values±s.e.m.

Results

The TiRP is a genetically engineered mouse melanoma model based on the tamoxifen-driven Cre-mediated expression of H-Ras^{G12V} and deletion of Ink4A/Arf in melanocytes, concomitantly with the expression of a specific tumor antigen of the MAGE-type, called HA. The TiRP model is characterized by tumors that are locally aggressive and insensitive to immunotherapies such as adoptive cell transfer (ACT). In particular, the TiRP model does not respond to the ACT of activated CD8⁺ T cells specific for the HA antigen (TCRP1A CD8⁺ T cells). The lack of response is explained by the fact that the transferred TCRP1A CD8⁺ T cells undergo apoptosis and disappear from the tumors in a few days. One of the main factors responsible for the immunoresistance of the TiRP tumors is the tumor enrichment in polymorphonuclear myeloid-derived suppressor cells (PMN-MDSC) that are able to induce apoptosis of tumor-infiltrating lymphocytes (TILs), e.g., tumor-infiltrating TCRP1A CD8⁺ T cells, through the Fas/Fas-ligand axis.

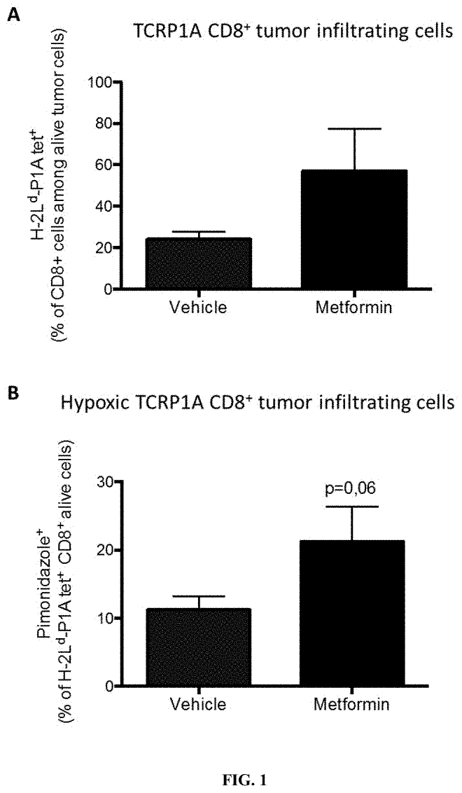

TiRP-tumor bearing mice were administered metformin concomitantly with the adoptive transfer of 10⁷ TCRP1A CD8⁺ T cells. As shown on FIG. 1A, 4 days after the ACT, the infiltration of TCRP1A CD8⁺ T cells in the tumor was significantly increased when the TiRP mice were administered metformin. In particular, administration of metformin allowed the recruitment of TCRP1A CD8⁺ T cells to the hypoxic regions of the tumor (FIG. 1B).

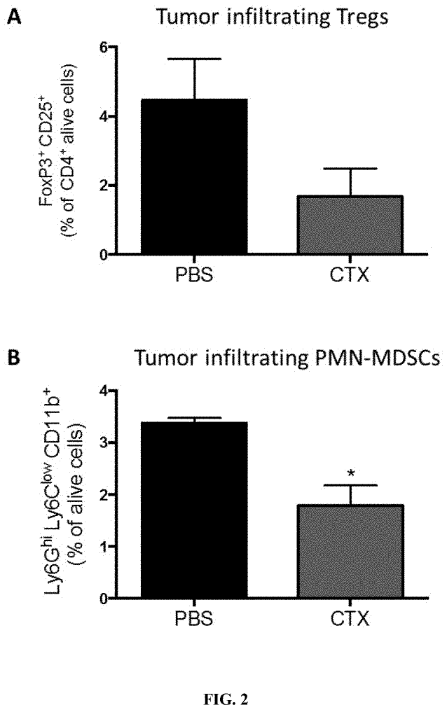

In parallel, a day before the adoptive transfer of 10⁷ TCRP1A CD8⁺ T cells, TiRP-tumor bearing mice were administered cyclophosphamide (CTX), an alkylating drug used for cancer treatment and known to have immunomodulatory effects. The effect of cyclophosphamide on different immune suppressive cells infiltrating the tumor was assessed. As shown on FIG. 2, the single injection of CTX to TiRP-tumor bearing mice reduced the tumor infiltration of Treg cells (FIG. 2A) and of PMN-MDSCs (FIG. 2B).

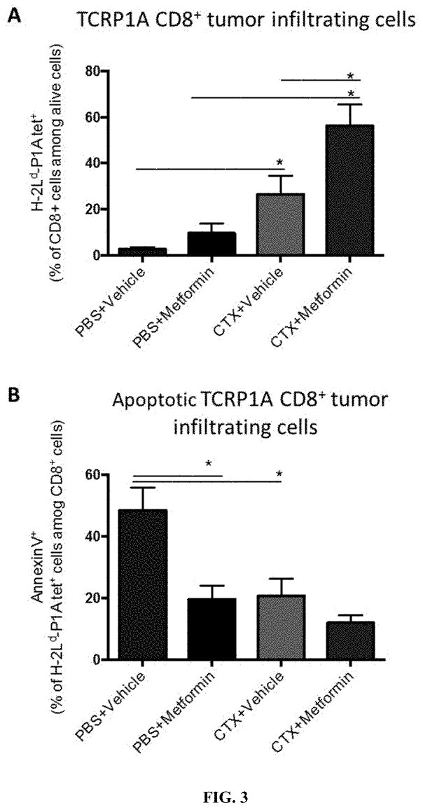

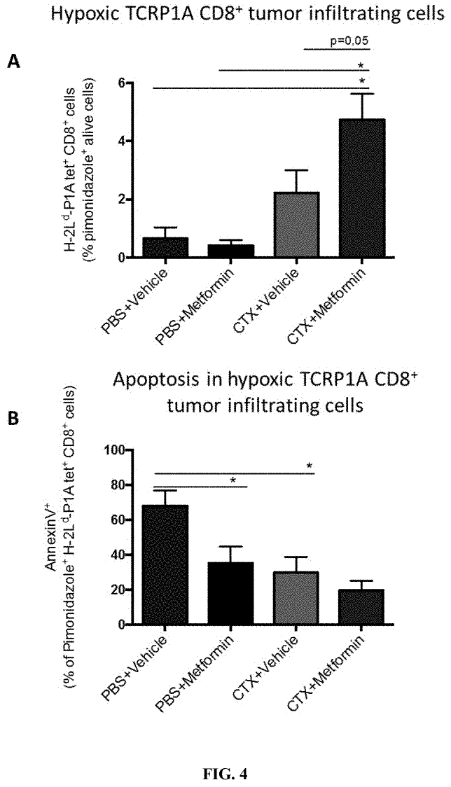

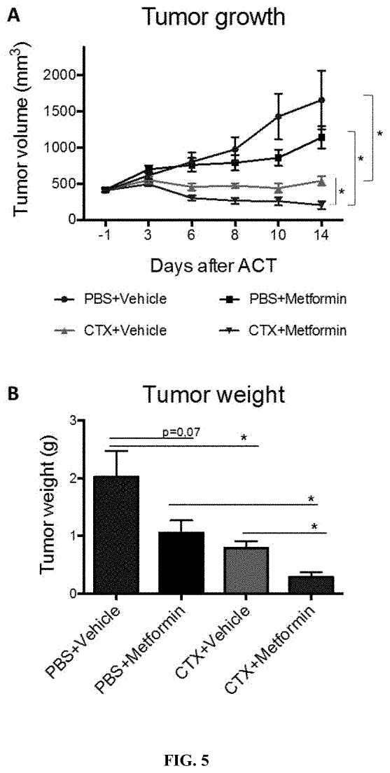

Next, the effect of the combined administration of metformin and cyclophosphamide was compared to the effect of metformin alone and of cyclophosphamide alone in TiRP-tumor bearing mice, 14 days after the adoptive cell transfer of TCRP1A CD8⁺ T cells. As previously observed, administration of metformin concomitantly with the adoptive transfer of 10⁷ TCRP1A CD8+ T cells increased tumor infiltration (FIG. 3A). Administration of metformin also increased survival of the transferred TCRP1A CD8+ T cells (FIG. 3B). An increase in both the infiltration and survival of the transferred TCRP1A CD8+ T cells was also observed after a single injection of cyclophosphamide (FIGS. 3A-B). Strikingly, when the single injection of cyclophosphamide was combined with the administration of metformin, the effect on both the infiltration and survival of the transferred TCRP1A CD8+ T cells was significantly increased (FIGS. 3A-B). Notably, cyclophosphamide and metformin acted in synergy and induced a marked increase of the infiltration of TCRP1A CD8+ T cells in the tumor. As shown on FIG. 4, the combined administration of cyclophosphamide and metformin also resulted in a significant TCRP1A CD8⁺ T cell infiltration (FIG. 4A) and survival (FIG. 4B) in hypoxic regions of the tumor. As previously observed, the effect of cyclophosphamide and metformin together was markedly higher than the effect of cyclophosphamide alone and than that of metformin alone.

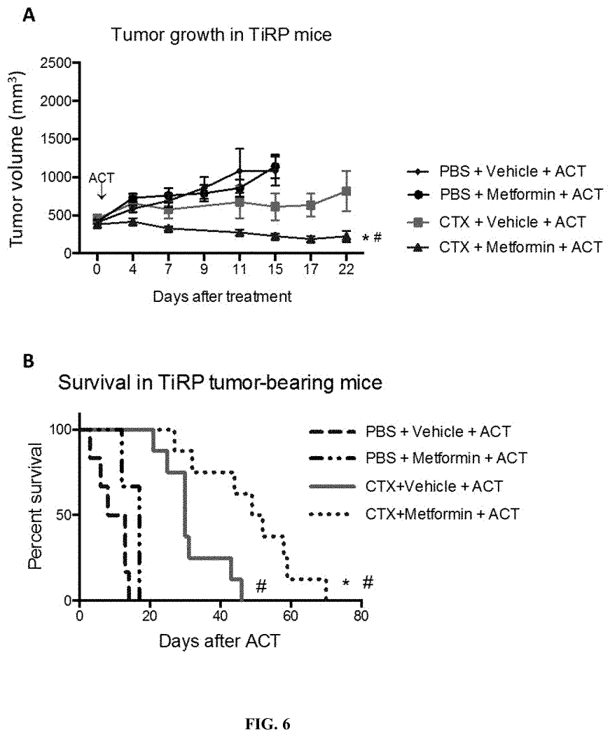

The effect of metformin and cyclophosphamide, alone or together, was also assessed on the tumor growth in the TiRP mice (FIG. 5A) and on the tumor weight 14 days after ACT (FIG. 5B). Administration of metformin for 14 days after ACT seemed to partially reduce tumor growth in TiRP mice. An injection of cyclophosphamide the day before ACT induced a tumor response to the immunotherapy and thus allowed an inhibition of the tumor growth. Strikingly, the combination of cyclophosphamide and metformin significantly enhanced the ACT-mediated tumor rejection in TiRP mice.

The effect of metformin and cyclophosphamide, alone or together, was next assessed on the tumor growth (FIG. 6A) and on TiRP mice survival (FIG. 6B). As previously observed, the combination of cyclophosphamide and metformin allowed a better control of tumor growth, compared either to metformin alone or to cyclophosphamide alone. Indeed, treatment with metformin alone did not seem to induce a significant tumor response to the immunotherapy while treatment with cyclophosphamide did induce a significant tumor response to the immunotherapy. However, when combined together, metformin and cyclophosphamide resulted in a marked increase of the tumor response to the immunotherapy. The inhibition of tumor growth was reflected on TiRP mice survival, with the combination of metformin and cyclophosphamide allowing a longer survival, notably in comparison to the effect of metformin alone and of cyclophosphamide alone.

Finally, to verify whether the efficacy of the combination of metformin and cyclophosphamide requires an adaptive immune response or whether metformin and cyclophosphamide are mainly affecting tumor cells themselves, tumor growth was analyzed in TiRP-tumor bearing mice treated with or without cyclophosphamide and/or metformin, in absence of ACT. As show in FIG. 7, cyclophosphamide significantly reduced tumor growth in TiRP mice even in absence of ACT, although tumor response was improved by the immunotherapy. By contrast, metformin alone did not inhibit tumor growth, either in the absence or in the presence of ACT. These results suggest that the additive effect of metformin observed in mice receiving the combination of metformin and cyclophosphamide together with ACT is mainly due to the effect of metformin on the infiltration of the transferred CD8⁺ T cells.

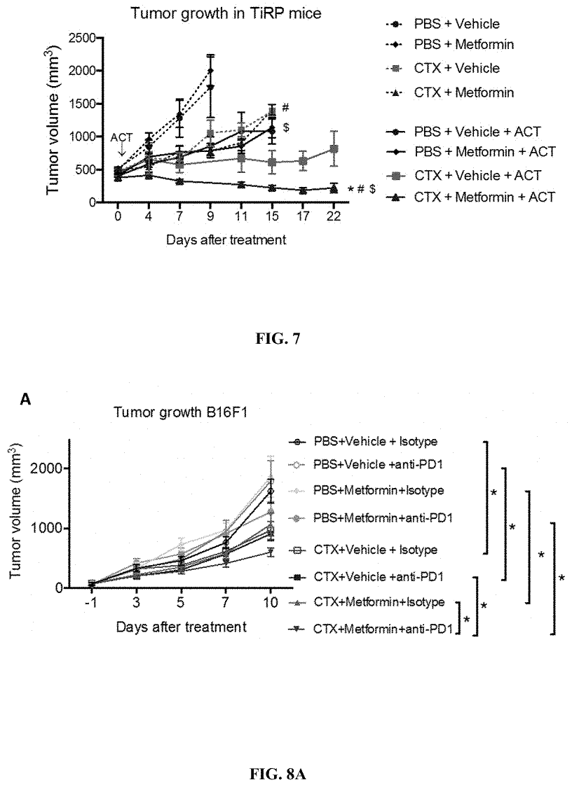

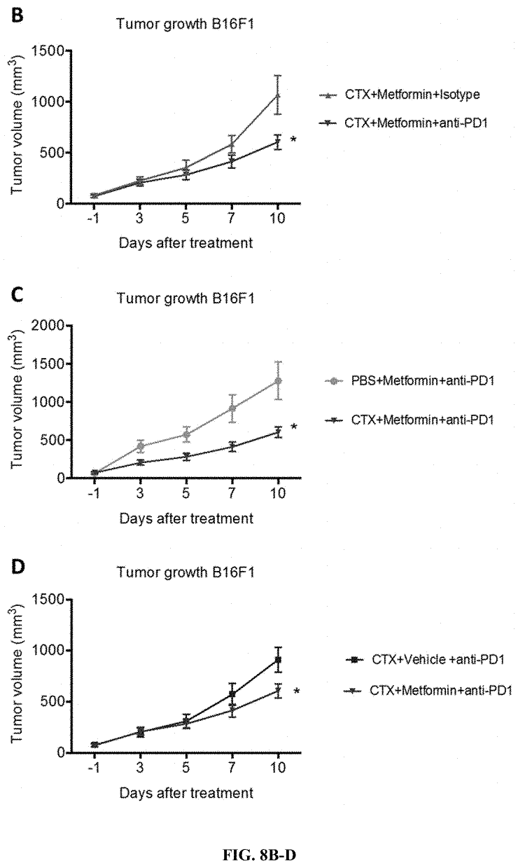

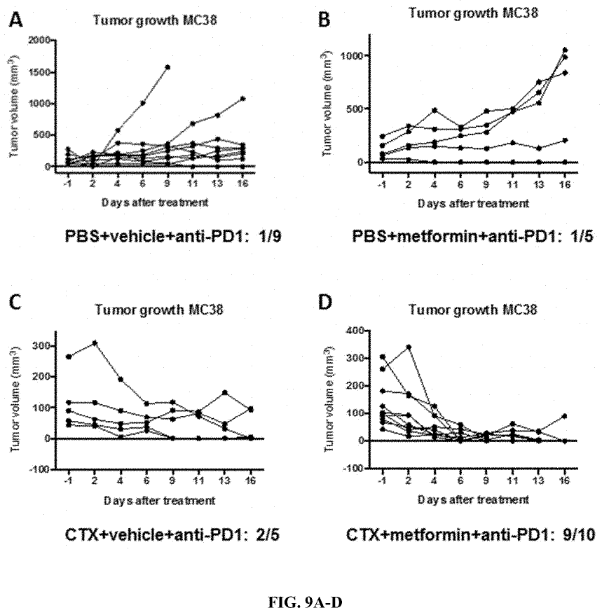

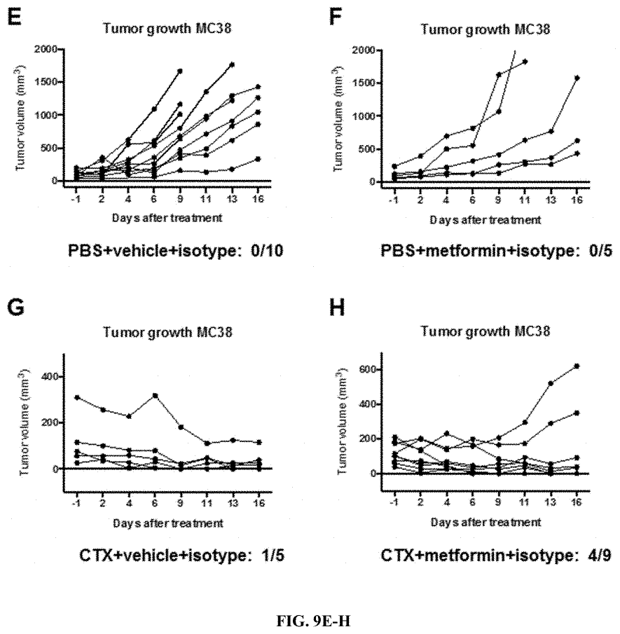

As shown on FIGS. 8 and 9, the effect of metformin and cyclophosphamide (alone or combined) on tumor growth was also assessed in association with a PD-1 inhibitor in transplanted mouse tumor models (melanoma and colon carcinoma).

The growth of B16F1 melanoma tumors was not significantly affected by a single treatment with either anti-PD-1 antibody or metformin (FIG. 8A). Interestingly, the checkpoint inhibitor therapy significantly reduced tumor growth when associated with the combination of metformin and cyclophosphamide (FIG. 8B). As observed with the adoptive cell therapy, the combination of cyclophosphamide and metformin associated with the checkpoint inhibitor therapy allowed a better control of tumor growth, compared to the association of the checkpoint inhibitor therapy either with metformin alone (see FIG. 8C) or with cyclophosphamide alone (see FIG. 8D). Treatment with metformin alone did not seem to induce a significant tumor response to the PD-1 inhibitor while treatment with cyclophosphamide induced per se a significant tumor response (see FIG. 8A). However, when combined together, metformin and cyclophosphamide resulted in a significant increase of the tumor response to the PD-1 inhibitor.

The effect of the combination of metformin and cyclophosphamide (CTX) on the tumor response to the anti-PD-1 checkpoint inhibitor therapy was also assessed in the MC38 mouse colon carcinoma model (FIG. 9). The individual tumor growth for each mouse after administration of metformin and cyclophosphamide (alone or combined) in association with a PD-1 inhibitor is shown on FIGS. 9A-D. Similarly, the individual tumor growth for each mouse after administration of metformin and cyclophosphamide (alone or combined) in association with the isotype control is shown on FIGS. 9E-H. Strikingly, 16 days after treatment with the PD-1 inhibitor associated with the combination of metformin and cyclophosphamide, 90% of the mice had rejected the tumor (FIG. 9D). By contrast, 16 days after treatment with the PD-1 inhibitor associated with metformin alone, only 20% of the mice had rejected the tumor (FIG. 9B) while 40% of the mice had rejected the tumor 16 days after treatment with the PD-1 inhibitor associated with cyclophosphamide alone (FIG. 9C).

Taken all together, the results presented above demonstrate that the combination of metformin and cyclophosphamide used with an immunotherapy, in particular an immunotherapy stimulating the T cell response, significantly improves the tumor response to said immunotherapy.

1-15. (canceled)

16. A method for treating a solid cancer in a subject in need thereof, said method comprising administering to the subject an immunotherapy and a combination comprising metformin and cyclophosphamide.

17. The method according to claim 16, wherein said combination comprising metformin and cyclophosphamide is used as an adjuvant for the immunotherapy.

18. The method according to claim 16, wherein said solid cancer is selected from the group comprising melanoma, Merkel cell skin cancer, lung cancer, head and neck cancer, bladder cancer, colon cancer, kidney cancer, cervical cancer, pancreatic cancer, prostate cancer, breast cancer, gastric cancer, and glioblastoma.

19. The method according to claim 16, wherein metformin and cyclophosphamide are administered sequentially.

20. The method according to claim 16, wherein metformin and cyclophosphamide are administered sequentially, with cyclophosphamide administered prior to the immunotherapy and metformin administered prior to or concomitantly with the immunotherapy.

21. The method according to claim 16, wherein metformin is administered at a dose ranging from about 0.15 mg per kilo body weight per day (mg/kg/day) to about 150 mg/kg/day and cyclophosphamide is administered at a dose ranging from about 0.15 mg/kg/day to about 150 mg/kg/day.

22. The method according to claim 16, wherein said immunotherapy comprises the adoptive transfer of immune cells or a checkpoint inhibitor.

23. The method according to claim 16, wherein said immunotherapy comprises the adoptive transfer of T cells.

24. The method according to claim 23, wherein said T cells are CD8⁺ T cells.

25. The method according to claim 23, wherein said T cells are autologous T cells.

26. The method according to claim 23, wherein said T cells are CAR T cells.

27. The method according to claim 23, wherein metformin and cyclophosphamide are administered sequentially, with cyclophosphamide administered prior to the adoptive transfer of T cells, and metformin administered prior to or concomitantly with the adoptive transfer of T cells.

28. The method according to claim 16, wherein said immunotherapy comprises a checkpoint inhibitor.

29. The method according to claim 28, wherein said checkpoint inhibitor is selected from the group comprising inhibitors of PD-1, inhibitors of PD-L1, inhibitors of CTLA-4, and any mixtures thereof.

30. The method according to claim 28, wherein said checkpoint inhibitor is an anti-PD-1 antibody.

31. The method according to claim 28, wherein said checkpoint inhibitor is an anti-PD-1 antibody selected from the group comprising pembrolizumab, nivolumab, cemiplimab, tislelizumab and spartalizumab.

32. The method according to claim 28, wherein said checkpoint inhibitor is an inhibitor of PD-L1 selected from the group comprising avelumab, atezolizumab and durvalumab.

33. The method according to claim 28, wherein said checkpoint inhibitor is an inhibitor of CTLA-4 selected from the group comprising ipilimumab and tremelimumab.

34. The method according to claim 28, wherein metformin and cyclophosphamide are administered sequentially, with cyclophosphamide administered prior to the checkpoint inhibitor and metformin administered prior to or concomitantly with the checkpoint inhibitor.

35. A method for potentiating an immunotherapy in a subject treated for a solid cancer with an immunotherapy, said method comprising administering to the subject a combination comprising metformin and cyclophosphamide.
